(19) United States
(12) Patent Application Publication
(10) Pub. No.: US 2011/0236399 A1
(43) Pub. Date: Sep. 29, 2011

(54) COMPOSITIONS AND METHODS FOR PROPHYLACTIC AND THERAPEUTIC TREATMENT OF INFECTION

(75) Inventors: James J. Mond, Silver Spring, MD (US); Irwin Scher, Wynnewood, PA (US); John F. Kokai-Kun, Frederick, MD (US)

(73) Assignee: BIOSYNEXUS INCORPORATED, Gaithersburg, MD (US)

(21) Appl. No.: 13/074,919

(22) Filed: Mar. 29, 2011

Related U.S. Application Data
(60) Provisional application No. 61/444,491, filed on Feb. 18, 2011.
(60) Provisional application No. 61/318,650, filed on Mar. 29, 2010.

(51) Int. Cl. A61K 39/395; C07K 16/12; A61P 31/04

(52) U.S. Cl. 424/164.1; 530/389.5

(57) ABSTRACT

This invention is directed to compositions and methods for treating (e.g., prophylactically and/or therapeutically) infection in a preterm infant. In particular, the invention provides anti-lipoteichoic acid (LTA) antibody compositions and methods of administering the same to a preterm infant (e.g., a low birth weight preterm infant (e.g., a very low birth weight preterm infant)) under conditions to establish anti-LTA antibody serum concentrations effective to kill and/or prevent growth of bacteria (e.g., Staphylococci). Compositions and methods of the invention find use in, among other things, clinical (e.g. therapeutic and preventative medicine) and research applications.

### CROSS REFERENCE TO RELATED APPLICATIONS

The present Application claims priority to U.S. Provisional Application Ser. No. 61/444,491 filed Feb. 18, 2011 and U.S. Provisional Application Ser. No. 61/318,650 filed Mar. 29, 2010, the entirety of each of which is herein incorporated by reference.

### FIELD OF THE INVENTION

This invention is directed to compositions and methods for treating (e.g., prophylactically and/or therapeutically) infection in a subject. In particular, the invention provides anti-lipoteichoic acid (LTA) antibody compositions and methods of administering the same to subject (e.g., an adult or infant (e.g., a preterm infant (e.g., a low birth weight preterm infant (e.g., a very low birth weight preterm infant)))) under conditions to establish anti-LTA antibody serum concentrations effective to kill and/or prevent growth of bacteria (e.g., Staphylococci). Compositions and methods of the invention find use in, among other things, clinical (e.g. therapeutic and preventative medicine) and research applications.

### BACKGROUND OF THE INVENTION

Of the about 4 million live births/yr in the U.S., approximately 8% (320,000) are prematurely born. Low birth weight (e.g., less than 2500 grams) accounts for seven percent of all births in the United States and is an important factor associated with infant morbidity and mortality (See, e.g., National Center for Health Statistics, Healthy People 2000: Maternal and Infant Heath Progress Review, 1999). Neonatal infection (e.g., sepsis (e.g., caused by infection with bacteria of the genus Staphylococcus)) is a complication experienced by a large percentage of low birth weight neonates and leads to significant levels of morbidity and mortality.

### SUMMARY OF THE INVENTION

This invention is directed to compositions and methods for treating (e.g., prophylactically and/or therapeutically) infection in a subject. In particular, the invention provides anti-lipoteichoic acid (LTA) antibody compositions and methods of administering the same to subject (e.g., an adult or infant (e.g., a preterm infant (e.g., a low birth weight preterm infant (e.g., a very low birth weight preterm infant)))) under conditions to establish anti-LTA antibody serum concentrations effective to kill and/or prevent growth of bacteria (e.g., Staphylococci).

Accordingly, the invention provides a method for treating (e.g., prophylactically or therapeutically treating) infection in a subject (e.g., adult, middle aged or infant (e.g., preterm infant)). In some embodiments, the invention provides an anti-LTA antibody composition comprising anti-LTA antibodies. In some embodiments, the invention provides an anti-LTA binding molecule composition comprising anti-LTA binding molecules. The present invention is not limited by the type of anti-LTA antibodies utilized. Indeed, a variety of anti-LTA antibodies may be used including, but not limited to, antibodies that bind to LTA with a binding affinity greater than (or equal to) about 10⁶ M⁻¹, 10⁷ M⁻¹, 10⁸M⁻¹, 10⁹ M, or 10¹⁰, (including affinities intermediate of these values). In some embodiments, the an anti-LTA binding molecule compositions is utilized in lieu of or together with an anti-LTA antibody composition. For example, in some embodiments, an anti-LTA binding molecules derived from an anti-LTA antibody is used which differs by one or more amino acid residues in the CDRs from the anti-LTA antibody parent and specifically binds LTA. In some embodiments, the anti-LTA antibody is a chimeric IgG1 antibody derived from a murine monoclonal antibody, 96-110 (A110). In some embodiments, the anti-LTA antibody is a chimeric IgG1 antibody derived from a murine monoclonal antibody, 96-120 (A120), Murine 96-110 is a murine IgG1 antibody, isolated from a mouse immunized with whole S. epidermidis strain Hay (deposited with the American Type Culture Collection (ATCC) on Dec. 19, 1990, under accession number 55133). Its isolation and anti-staphylococcal properties have been described in U.S. Pat. No. 6,610,293, the entire contents of which are herein incorporated by reference. Murine 96-110 was found to specifically bind lipoteichoic acid, a major constituent of the cell wall of Gram positive bacteria. The hybridoma cell line which produces 96-110 was deposited on Jun. 13, 1997, with the ATCC according to the provisions of the Budapest Treaty and was assigned ATCC accession number HB-12368. Chimeric LTA antibody, A120, is described in U.S. Pat. No. 7,250,494, the entire contents of which are herein incorporated by reference. As described in U.S. Pat. Nos. 6,610,293 and 7,250,494, the A110 and A120 antibodies have been shown to bind and opsonize whole Gram positive bacteria, including multiple strains of S. epidermidis and S. aureus, thereby enhancing phagocytosis and killing of such bacteria in vitro and enhancing protection from lethal infection of such bacteria in vivo. Although an understanding of the mechanism is not needed to practice the invention and the invention is not limited to any particular mechanism, in some embodiments, the anti-LTA binding molecules and/or anti-LTA antibodies of the invention inhibits the interaction between LTA, a major constituent on the surface of Gram positive bacteria, and its receptor, toll-like receptor 2 (TLR2), on phagocytic cells, e.g., macrophages and neutrophils, which reduces LTA-mediated cytokine production. In some embodiments, anti-LTA antibodies and/or binding molecules of the invention selectively recognize and bind to all Gram positive bacteria and do not recognize or bind to Gram negative bacteria. In some embodiments, anti-LTA antibody compositions and/or binding molecule compositions of the invention bind to multiple serotypes of S. epidermidis, S. epidermidis strain Hay, S. hemolyticus, S. hominus and multiple serotypes of S. aureus. In some embodiments, anti-LTA antibody compositions and/or binding molecule compositions of the invention are opsonic, thereby enhancing and/or leading to clearance of bacteria from tissues and blood (e.g., in an adult or infant (e.g., preterm infant (e.g., LBW infant (e.g., VLBW infant)))). In some embodiments anti-LTA antibody compositions and/or binding molecule compositions of the invention provide protection against infection caused by Gram positive bacteria in a preterm infant (e.g., LBW infant (e.g., VLBW infant)). In some embodiments, anti-LTA antibody compositions and/or binding molecule compositions of the invention are administered to a subject (e.g., adult or infant (preterm infant (e.g., LBW infant (e.g., VLBW infant)))) in order to treat (e.g., therapeutically and/or prophylactically) infection and/or sepsis in the subject.

In some embodiments, the invention provides anti-LTA antibody composition and/or binding molecule composition dosing regimens that produce serum concentrations within a preterm infant (e.g., LBW infant (e.g., VLBW infant) at an effective level (e.g., compared to the serum concentration of anti-LTA antibody composition and/or anti-LTA binding molecule composition required in a full term infant that promotes clearance and/or killing of bacteria) with minimal to no adverse effects to the infant subject. For example, in some embodiments, an infant (e.g., preterm infant (e.g., LBW infant) subject is administered about 100 mg anti-LTA antibody per kilogram (kg) of the subject. In some embodiments, an infant (e.g., preterm infant (e.g., LBW infant) subject is administered about 100 mg anti-LTA antibody per kilogram (kg) of the subject on three consecutive days (e.g., days 1, 2, and 3). In some embodiments, an infant (e.g., preterm infant (e.g., LBW infant) subject is administered 100 mg anti-LTA antibody per kilogram (kg) of the subject on three or more non-consecutive days. In a preferred embodiment, an anti-LTA antibody composition is administered (e.g., infused in) to a LBW infant (e.g., preterm infant (e.g., VLBW infant)) at about 100 mg anti-LTA antibody per kilogram (kg) infant weight daily for 3 days followed by administration (e.g., infusion) of about 100 mg anti-LTA antibody per kg infant weight on days 9, 16 and 23.

In a preferred embodiment, the serum concentration of anti-LTA binding molecule and/or anti-LTA antibody needed in order to provide protection from and/or killing of bacteria in a preterm infant (e.g., low birth weight infant (e.g., very low birth weight infant) is more than that needed in order to provide protection from and/or killing of bacteria in a full term infant. Thus, in some embodiments, the invention provides a method of administering an anti-LTA antibody and/or anti-LTA binding molecule to a preterm infant (e.g., low birth weight infant (e.g., very low birth weight infant) that results in a higher effective serum concentration of the anti-LTA antibody and/or anti-LTA binding molecule than that required in a full term infant. For example, in some embodiments, the dosing regimen produces an infant anti-LTA antibody and/or anti-LTA binding molecule serum level that is ≧100 μg/ml, ≧200 μg/ml, ≧300 μg/ml, ≧350 μg/ml, ≧400 μg/ml, ≧450 μg/ml, ≧500 μg/ml, ≧550 μg/ml, ≧600 μg/ml, ≧650 μg/ml, ≧700 μg/ml, ≧750 μg/ml, ≧800 μg/ml, or higher. In some embodiments, the dosing regimen produces an infant anti-LTA antibody and/or anti-LTA binding molecule serum level that are ≧500 μg/ml. In some embodiments, infant anti-LTA antibody serum levels (e.g., of ≧500 μg/ml) are maintained for one or more (e.g., 2, 3, 4, 5, 6, 7, 8, 9, 10, 11, 12, 13, 14, 15, 16, 17, 18, 19, 20, 21, 22, 23, 24 or more) days. The invention is not limited by the route of administration of an anti-LTA antibody composition or anti-LTA binding molecule composition. For example, in some embodiments the composition is administered intravenously, intramuscularly, intraperitoneally, intradermally, subcutaneously or via another route of administration described herein. In some embodiments, anti-LTA antibody composition or anti-LTA binding molecule composition is administered via intravenous infusion at 0.01 ml per kilogram infant weight per minute. The invention is also not limited by the type of bacteria treated (e.g., therapeutically and/or prophylactically) using a composition comprising anti-LTA antibody composition or anti-LTA binding molecule composition of the invention. For example, in some embodiments, type of gram positive bacteria expressing LTA can be treated including, but not limited to, S. epidermidis, S. epidermidis strain Hay, S. hemolyticus, S. hominus and serotypes of S. aureus. In some embodiments, bacterial growth and/or infection (e.g., sepsis) is prevented and/or treated in infants receiving anti-LTA antibody therapy. In some embodiments, the method comprises administration of an anti-LTA antibody composition and one or more other antibacterial agents. The present invention is not limited to the type of anti-bacterial agent co-administered with an anti-LTA antibody composition. In some embodiments, the antibacterial agent is a non-anti-LTA antibody, an antibiotic or other biologically active (e.g., antibacterial agent). In some embodiments, an anti-LTA antibody composition is co-administered with one or more antibiotics. For example, one or more antibiotics may be administered with, before and/or after administration of an anti-LTA antibody composition.

The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines. In some embodiments, an anti-LTA antibody composition is co-administered with intravenous fluids. In some embodiments, an anti-LTA antibody composition is co-administered with a vasopressor. In some embodiment, anti-LTA-antibody composition or anti-LTA binding molecule composition is co-administered with other treatments such as surgical drainage of infected fluid collections, fluid replacement and/or appropriate support for organ dysfunction (e.g., including, but not limited to, hemodialysis (e.g., for kidney failure), mechanical ventilation (e.g., in pulmonary dysfunction), transfusion of blood products, drug and fluid therapy for circulatory failure and/or parenteral nutrition. In some embodiments, the invention provides anti-LTA antibody composition and/or binding molecule composition comprising a pharmaceutically acceptable carrier (e.g., a pharmaceutical composition comprising an anti-LTA antibody). The present invention is not limited by the type of pharmaceutically acceptable carrier utilized. Indeed, compositions useful in a pharmaceutical formulation are well known in the art and can include any one or more of the types of carries described herein.

In one embodiment, the invention provides the use of an anti-LTA antibody composition and/or anti-LTA binding molecule composition in therapy. In another embodiment, the invention provides the use of an anti-LTA antibody composition and/or anti-LTA binding molecule composition in the manufacture of a medicament for the treatment of a disease or disorder associated with a Gram positive bacterial infection (e.g., a S. aureus bacterial infection, a S. epidermidis bacterial infection, a coagulase negative staphylococci bacterial infection, or a Streptococcus mutans bacterial infection).

In one embodiment, therapy (e.g., therapeutic and/or prophylactic therapy) occurs within 6 hours after birth. In one embodiment, therapy occurs within 12 hours after birth. In one embodiment, therapy occurs within 24 hours after birth. In one embodiment, therapy occurs within 36 hours after birth. In one embodiment, therapy occurs within 6 hours after 48. In one embodiment, therapy occurs within 60 hours after birth. In another embodiment, therapy occurs within 96 hours after birth. In another embodiment initiation of anti-LTA antibody therapy occurs within 3 to 14 days after birth. In one embodiment, the anti-LTA antibody composition is administered intravenously. In another embodiment administration of anti-LTA antibody therapy is initiated at the same time as another type of therapy (e.g., antibiotic therapy).

In one embodiment, the low birth weight preterm infant weighs 2500 grams or less. In another embodiment, the low birth weight preterm infant weighs 1500 grams or less. In another embodiment, the low birth weight preterm infant weighs 1000 grams or less. In another embodiment, the low birth weight preterm infant weighs 850 grams or less.

The invention also provides a kit for preventing and/or treating bacterial growth and/or infection (e.g., sepsis) in a low birth weight preterm infant, wherein the kit comprises an anti-LTA antibody composition and/or anti-LTA binding molecule composition and an instructional material teaching the indications, dosage and schedule of administration for the anti-LTA antibody composition or anti-LTA binding molecule composition, in a dose effective for preventing and/or treating bacterial growth and/or infection (e.g., sepsis) within the infant.

### BRIEF DESCRIPTION OF THE DRAWINGS

The following figures form part of the present specification and are included to further demonstrate certain aspects and embodiments of the present invention. The invention may be better understood by reference to one or more of these figures in combination with the description of specific embodiments presented herein.

FIG. 1 shows the concentration (μg/ml) of anti-LTA antibody composition required for opsonic activity (percent killing) in a model representing VLBW immune systems (HL-60s+C1q) is at least 10 fold higher than that in the FDA approved model representing term infant and/or adult immune system (polymorphonuclear cells (PMNs)+C1q or PMNs+complement, respectively).

FIG. 2 shows a table of patient baseline characteristics by treatment group.

FIG. 3 shows a graph of plasma anti-LTA antibody concentrations over time by PAGIBAXIMAB dose group on a semilogarithmic axis.

FIG. 4 shows a table of Adverse events occurring in 10% of patients in the intent-to-treat population by treatment group.

FIG. 5 shows a table of opsonophagoctic activity (bacterial killing) in serum of neonates over time against S. epidermidis ATCC strain 55133.

FIG. 6 shows PFGE patterns for genetic relatedness of the 25 CONS isolates.

FIG. 7 shows a Two Compartment Model used to describe the concentration time course of anti-LTA antibody composition.

FIG. 8 shows the observed and simulated concentration time profiles for patients with weight Less than 800 g.

FIG. 9 shows observed and simulated concentration time profiles for patients with weight 800 g to 1000 g.

FIG. 10 shows observed and simulated concentration time profiles for patients with weight 1000 g or higher.

FIG. 11 shows observed, individual and typical predicted concentrations versus time.

FIG. 12 shows observed versus predicted concentrations.

FIG. 13 shows observed versus individual predicted concentrations.

FIG. 14 shows observed versus typical predicted concentrations.

FIG. 15 shows observed versus typical predicted concentrations.

### DEFINITIONS

To facilitate an understanding of the present invention, a number of terms and phrases are defined below:

The term “preterm infant” refers to an infant born before 37 weeks of gestation. This includes terms such as premature infant or preemie. The terms “low birth weight preterm infant,” “low birth weight infant, and “LBW infant” refer to a preterm infant weighing less than 2,500 grams at birth. This term includes preterm infants described as low birth weight (less than 2,500 grams), very low birth weight (less than 1,500 grams) and extremely low birth weight (less than 1,000 grams).

The terms “LTA antibody” and “anti-LTA” are used interchangeably herein to refer to an antibody that binds to one or more epitopes or antigenic determinants within lipoteichoic acid, a constituent found on Gram positive bacteria.

As used herein, the terms “optimized antibody” and “mutant antibody,” used interchangeably herein, refer to an antibody having at least one amino acid which is different from the parent antibody in at least one complementarity determining region (CDR) in the light or heavy chain variable region, which confers a higher binding affinity, e.g., a 2-fold or more fold higher binding affinity, to the binding antigen as compared to the parent antibody.

As used herein, the term “LTA binding molecule” or “lipoteichoic acid binding molecule” refers to a molecule which specifically binds to one or more epitopes or antigenic determinants within lipoteichoic acid (LTA), a constituent found on Gram positive bacteria.

In one embodiment, an LTA binding molecule is a whole antibody. In another embodiment, an LTA binding molecule is an antibody fragment. In one embodiment, an LTA binding molecule is a humanized antibody. In another embodiment, an LTA binding molecule is a human antibody. In another embodiment, an LTA binding molecule is a single chain antibody. In another embodiment, an LTA binding molecule is an immunoconjugate. In another embodiment, an LTA binding molecule is a defucosylated antibody. In yet another embodiment, an LTA binding molecule is a bispecific antibody. In another embodiment, an LTA binding molecule is an aglycosylated antibody. As used herein “anti-LTA binding molecule composition” and “anti-LTA antibody composition” refer to composition comprising an anti-LTA binding molecule or anti-LTA antibody, respectively.

The term “antibody” or “immunoglobulin” as used interchangeably herein, is intended to refer to proteins comprised of four polypeptide chains, two heavy (H) chains and two light (L) chains inter-connected by disulfide bonds, which has the ability to specifically bind antigen. Each heavy chain is comprised of a heavy chain variable region (abbreviated herein as HCVR or VH) and a heavy chain constant region. The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4. It is intended that the term “antibody” encompass any Ig class or any Ig subclass (e.g. the IgG1, IgG2, IgG3, and IgG4 subclasses of IgG) obtained from any source (e.g., in exemplary embodiments, humans and non-human primates, and in additional embodiments, mice, rodents, lagomorphs, caprines, bovines, equines, ovines, etc.). The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals.

The term “IgG subclass” refers to the four subclasses of immunoglobulin class IgG-IgG1, IgG2, IgG3, and IgG4 that have been identified in humans and higher mammals by the “(heavy chains of the immunoglobulins, “(1-”(4, respectively)).

As used herein, the terms “complementarity determining region” and “CDR” refer to the regions that are primarily responsible for antigen-binding. There are three CDRs in a light chain variable region (LCDR1, LCDR2, and LCDR3), and three CDRs in a heavy chain variable region (HCDR1, HCDR2, and HCDR3). The residues that make up these six CDRs have been characterized by Kabat and Chothia as follows: residues 24-34 (LCDR1), 50-56 (LCDR2) and 89-97 (LCDR3) in the light chain variable region and 31-35 (HCDR1), 50-65 (HCDR2) and 95-102 (HCDR3) in the heavy chain variable region; Kabat et al., (1991) Sequences of Proteins of Immunological Interest, 5th Ed. Public Health Service, National Institutes of Health, Bethesda, Md., herein incorporated by reference; and residues 26-32 (LCDR1), 50-52 (LCDR2) and 9196(LCDR3) in the light chain variable region and 26-32 (HCDR1), 53-55 (HCDR2) and 96-101 (HCDR3) in the heavy chain variable region; Chothia and Lesk (1987) J. Mol. Biol. 196: 901 917, herein incorporated by reference. Unless otherwise specified, the terms “complementarity determining region” and “CDR” as used herein, include the residues that encompass both the Kabat and Chothia definitions (i.e., residues 24-34 (LCDR1), 50-56 (LCDR2), and 89-97 (LCDR3) in the light chain variable region; and 26-35 (HCDR1), 50-65 (HCDR2), and 95-102 (HCDR3)). Also, unless specified, as used herein, the numbering of CDR residues is according to Kabat.

As used herein, the term “framework” refers to the residues of the variable region other than the CDR residues as defined herein. There are four separate framework sub-regions that make up the framework: FR1, FR2, FR3, and FR4. In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to Kabat.

As used herein, the term “fully human framework” means a framework with an amino acid sequence found naturally in humans. Examples of fully human frameworks, include, but are not limited to, KOL, NEWM, REI, EU, TUR, TE1, LAY and POM (See, e.g., Kabat et al., (1991) Sequences of Proteins of Immunological Interest, U.S. Department of Health and Human Services, NIH, USA; and Wu et al., (1970) J. Exp. Med. 132,211 250, both of which are herein incorporated by reference).

As used herein, the term “modify” or “modified amino acid” refers to an amino acid which is different or not the same as the corresponding amino acid residue in the parent A110 heavy chain variable region CDRs or the parent A110 light chain variable region CDRs. A modified amino acid may be any amino acid, including but not limited to, leu, met, ala, val, leu, ile, cys, ser, thr, asp, glu, asn, gln, his, lys, arg, gly, pro, trp, tyr, or phe.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein. Antibodies can exist in monomeric or polymeric form, for example, IgM antibodies which exist in pentameric form and/or IgA antibodies which exist in monomeric, dimeric or multimeric form.

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means.

Exemplary fragments include Fab, Fab′, F(ab′)2, Fabc, Fv, single chains and/or single-chain antibodies. The term “antigen-binding fragment” refers to a polypeptide fragment of an immunoglobulin or antibody that binds antigen or competes with intact antibody (i.e., with the intact antibody from which they were derived) for antigen binding (i.e., specific binding). Antigen-binding fragments can be produced by recombinant DNA techniques, or by enzymatic or chemical cleavage of intact immunoglobulins. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. In exemplary embodiments, the antibody exhibits no crossreactivity (e.g., does not crossreact with non-LTA constituents). An antibody that “does not exhibit significant crossreactivity” is one that will not appreciably bind to an undesirable entity. Specific binding can be determined according to any art-recognized means for determining such binding.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

As used herein, the term “avidity” refers to the strength of the antigen-antibody bond after formation of reversible complexes.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. See, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539 (incorporated by reference in their entirety for all purposes).

A “humanized immunoglobulin” or “humanized antibody” can be made using any of the methods described herein or those that are well known in the art.

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, e.g., a non-human mammal.

The term “significant identity” means that two polypeptide sequences, when optimally aligned, such as by the programs GAP or BESTFIT using default gap weights, share at least 60-70% sequence identity, more preferably at least 70-80% sequence identity, more preferably at least 80-90% identity, even more preferably at least 90-95% identity, and even more preferably at least 95% sequence identity or more (e.g., 99% sequence identity or more). The term “substantial identity” means that two polypeptide sequences, when optimally aligned, such as by the programs GAP or BESTFIT using default gap weights, share at least 80-90% sequence identity, preferably at least 90-95% sequence identity, and more preferably at least 95% sequence identity or more (e.g., 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Bioi. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (See, e.g., Ausubel et al., Current Protocols in Molecular Biology). One example of algorithm that is suitable for determining percent sequence identity and sequence similarity is the BLAST algorithm, which is described in Altschul et al., J. Mol. Bioi. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)). Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another class.

Accordingly, another aspect of the invention pertains to CDRs that contain changes in amino acid residues. In one embodiment, such CDRs are at least 70-95%, at least 80-95%, or preferably at least 90-95% identical to the amino acid sequence of a CDR sequence identified herein. In another embodiment, such CDRs are at least 40% identical, 50%, 60%, 70%, 80%, 90%, 91%, 92%, 93%, 94%, 95%, 96%, 97%, 98%, or 99% identical to the amino acid sequence of a CDR sequence identified herein.

Preferably, humanized immunoglobulins or antibodies bind antigen with an affinity that is within a factor of three, four, or five of that of the corresponding non-humanized antibody.

When describing the binding properties of an immunoglobulin or antibody chain, the chain can be described based on its ability to “direct antigen binding”. A chain is said to “direct antigen binding” when it confers upon an intact immunoglobulin or antibody (or antigen binding fragment thereof) a specific binding property or binding affinity. A mutation (e.g., a backmutation) is said to substantially affect the ability of a heavy or light chain to direct antigen binding if it affects (e.g., decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (e.g., decrease) the ability of a chain to direct antigen binding” if it affects (e.g., decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

An “antigen” is an entity to which an immunoglobulin or antibody (or antigen binding fragment thereof) specifically binds.

As used herein, the term “antigen binding site” refers to a site that specifically binds (immunoreacts with) an antigen (e.g., a cell surface or soluble antigen). Antibodies of the invention preferably comprise at least two antigen binding sites. An antigen binding site commonly includes immunoglobulin heavy chain and light chain CDRs and the binding site formed by these CDRs determines the specificity of the antibody. An “antigen binding region” or “antigen binding domain” is a region or domain (e.g., an antibody region or domain that includes an antibody binding site as defined herein).

As used herein, the term “immunotherapy” refers to a treatment, for example, a therapeutic or prophylactic treatment, of a disease or disorder intended to and/or producing an immune response (e.g., an active or passive immune response).

As used herein, the term “adjuvant” refers to any substance that can stimulate an immune response (e.g., a systemic immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present invention are administered with one or more adjuvants (e.g., to skew the immune response towards a Th1 or Th2 type response).

As used herein, the terms “nucleic acid sequence encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage, Therefore, an end of an oligonucleotide or polynucleotide, is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “codon” or “triplet” refers to a group of three adjacent nucleotide monomers which specify one of the naturally occurring amino acids found in polypeptides. The term also includes codons which do not specify any amino acid.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements. Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the terms “complementary” or “complementarity” are used in reference to polynucleotides (i.e., a sequence of nucleotides) related by the base-pairing rules. For example, the sequence “5′-A-G-T 3”, is complementary to the sequence “3-T-C-A-5′”. Complementarity may be “partial”, in which only some of the nucleic acids' bases are matched according to the base pairing rules, or, there may be “complete” or “total” complementarity between the nucleic acids. The degree of complementarity between nucleic acid strands has significant effects on the efficiency and strength of hybridization. This is of particular importance in amplification reactions, as well as in detection methods that depend upon binding between nucleic acids.

As used herein, the term “the complement of” a given sequence is used in reference to the sequence that is completely complementary to the sequence over its entire length. For example, the sequence 5′ A G T A 3′ is “the complement” of the sequence 3′-T-C-A-T-5′.

The term “homology” (when in reference to nucleic acid sequences) refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous”.

The term “treatment” as used herein, is defined as the application or administration of a therapeutic agent to a patient, or application or administration of a therapeutic agent to an isolated tissue or cell line from a patient, who has an infection or disease, a symptom of infection or disease or a predisposition toward an infection or disease, with the purpose to cure, heal, alleviate, relieve, alter, remedy, ameliorate, improve or affect the disease, the signs or symptoms of infection or disease or the predisposition toward infection or disease.

The term “effective dose” or “effective dosage” is defined as an amount sufficient to achieve or at least partially achieve the desired effect. The term “therapeutically effective dose” is defined as an amount sufficient to cure or at least partially arrest disease and its complications in a subject (e.g., preterm infant (e.g., low birth weight preterm infant)) already suffering from the disease. Amounts effective for this use depend upon the severity of the disease, the patient's general physiology, e.g., the patient's body mass, age, gender, the route of administration, and other factors well known to physicians and/or pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). In a preferred embodiment, an effective dose is that amount which produces a serum concentration level of at least 500 μg/ml in a subject (e.g., preterm infant (e.g., low birth weight preterm infant)). Thus, an effective dose of an antibody composition (e.g., anti-LTA antibody composition and/or anti-LTA binding molecule composition) used in the present compositions and methods will range, for example, between 1 μg/kg and 1 g/kg, preferably between 1 μg/kg and 500 mg/kg, even more preferably between 50 mg/kg and 350 mg/kg. Additional exemplary doses include, but are not limited to, 10 μg/kg, 30 μg/kg, 60 μg/kg, 90 μg/kg, 100 μg/kg, 200 μg/kg, 300 μg/kg, 500 μg/kg, 1 mg/kg, 30 mg/kg, 60 mg/kg, 90 mg/kg, 100 mg/kg, 110 mg/kg, 120 mg/kg, 125 mg/kg, 130 mg/kg, 140 mg/kg, 150 mg/kg, 160 mg/kg, 170 mg/kg, 175 mg/kg, 180 mg/kg, 190 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1 g/kg or more. Doses intermediate in the above ranges are also intended to be within the scope of the invention. In another embodiment, the effective dose can comprise multiple administrations of a dose. For example, the effective dose of 600 mg/kg may consist of the administration of a dose of 100 mg/kg on days 0, 1 and 2, and the administration of a dose of 100 mg/kg weekly thereafter for three weeks.

As used herein, the terms “administration,” “administer” and “administering” refer to the act of giving a composition of the present invention (e.g., an anti-LTA antibody composition)) to a subject. An exemplary route of administration is via a parenteral route, e.g., subcutaneous, intravenous or intraperitoneal administration. Additional exemplary routes of administration to the human body include, but are not limited to, through the eyes (ophthalmic), mouth (oral), skin (transdermal), nose (nasal), lungs (inhalant), oral mucosa (buccal), ear, rectal, by injection, topically, and the like.

As used herein, the terms “co-administration” and “co-administering” refer to the administration of at least two agent(s) (e.g., an anti-LTA antibody composition and one or more other agents or therapies (e.g., a composition comprising one or more antibiotics)) to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

The terms “host” or “subject,” as used herein, refer to an individual that is administered either prophylactic or therapeutic treatment with one or more compositions and/or methods of the invention. Subjects include, but are not limited to, mammals (e.g., murines, simians, equines, bovines, porcines, canines, felines, and the like), and most preferably includes humans (e.g., preterm infant (e.g., low birth weight infant (e.g., very low birth weight infant))). In the context of the invention, the term “subject” generally refers to an individual who will be administered or who has been administered one or more compositions of the present invention.

As used herein, the terms “subject” and “patient” refer to any animal, such as a mammal like a dog, cat, bird, livestock, and preferably a human.

The terms “animal model” or “model animal,” as used herein, include a member of a mammalian species such as rodents, non-human primates, sheep, dogs, and cows that exhibit features or characteristics of a certain system of disease or disorder, e.g., a human system, disease or disorder, e.g., a bacterial infection. Exemplary non-human animals selected from the rodent family include rabbits, guinea pigs, rats and mice, most preferably mice. An “animal model” of, or “model animal” having, a bacterial infection exhibits, for example, a Staphylococcal bacterial infection.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology of the invention, as described herein. In another embodiment, a “suitable control” or “appropriate control” is a value, level, feature, characteristic, property, etc. determined in a patient, e.g., a control or normal subject exhibiting, for example, normal traits. In yet another embodiment, a “suitable control” or “appropriate control” is a predefined value, level, feature, characteristic, property, etc.

The term “Fc immunoglobulin variant” or “Fc antibody variant” includes immunoglobulins or antibodies (e.g., humanized immunoglobulins, chimeric immunoglobulins, single chain antibodies, antibody fragments, etc.) having an altered Fc region. Fc regions can be altered, for example, such that the immunoglobulin has an altered effector function. In some embodiments, the Fc region includes one or more amino acid alterations in the hinge region. Antibodies including hinge region mutations can be made, as described in, for example, U.S. Pat. No. 5,624,821, and U.S. Pat. No. 5,648,60, incorporated by reference herein.

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life.

The term “effector molecule” refers to a molecule that is capable of binding to the Fc region of an antibody (e.g., an IgG antibody) including, but not limited to, a complement protein or a Fc receptor. The term “effector cell” refers to a cell capable of binding to the Fc portion of an antibody (e.g., an IgG antibody) typically via an Fc receptor expressed on the surface of the effector cell including, but not limited to, lymphocytes, e.g., antigen presenting cells and T cells.

The term “Fc region” refers to a C-terminal region of an IgG antibody, in particular, the C-terminal region of the heavy chain(s) of said IgG antibody. Although the boundaries of the Fc region of an IgG heavy chain can vary slightly, a Fc region is typically defined as spanning from about amino acid residue Cys226 to the carboxylterminus of a human IgG heavy chain(s).

The term “aglycosylated” antibody refers to an antibody lacking one or more carbohydrates by virtue of a chemical or enzymatic process, mutation of one or more glycosylation sites, expression in bacteria, etc. An aglycosylated antibody may be a deglycosylated antibody, that is an antibody for which the Fc carbohydrate has been removed, for example, chemically or enzymatically. Alternatively, the aglycosylated antibody may be a nonglycosylated or unglycosylated antibody, that is an antibody that was expressed without Fc carbohydrate, for example by mutation of one or more residues that encode the glycosylation pattern or by expression in an organism that does not attach carbohydrates to proteins, for example bacteria.

“Kabat numbering” unless otherwise stated, is as taught in Kabat et al. (Sequences of Proteins of Immunological Interest, 5th Ed. Public Health Service, National Institutes of Health, Bethesda, Md. (1991)), expressly incorporated herein by reference. “ED numbering” unless otherwise stated, is also taught in Kabat et al. (Sequences of Proteins of Immunological Interest, 5th Ed. Public Health Service, National Institutes of Health, Bethesda, Md. (1991)) and, for example, refers to the numbering of the residues in heavy chain antibody sequences using the ED index as described therein. This numbering system is based on the sequence of the Eu antibody described in Edelman et al., 63(1):78-85 (1969).

The term “Fc receptor” or “FcR” refers to a receptor that binds to the Fc region of an antibody. Typical Fc receptors that bind to an Fc region of an antibody (e.g., an IgG antibody) are described in Ravetch and Kinet, Annu. Rev. Immunol9:457-92 (1991); Capel et al., Immunomethods 4:25-34 (1994); and de Haas et al., J. Lab. Clin. Med. 126:330-41 (1995), hereby incorporated by reference in its entirety.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

A used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “enhanced immunity” refers to an increase in the level of adaptive and/or acquired immunity in a subject to a given immunogen (e.g., microorganism (e.g., pathogen)) following administration of a composition (e.g., an anti-LTA antibody composition) relative to the level of adaptive and/or acquired immunity in a subject that has not been administered the composition.

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., shortened gestational period). Thus, it is not intended that the present invention be limited to any particular risk (e.g., a subject may be “at risk for disease” simply by being born prematurely nor is it intended that the present invention be limited to any particular disease.

As used herein, the term “kit” refers to any delivery system for delivering materials. In the context of anti-LTA antibody compositions, such delivery systems include systems that allow for the storage, transport, or delivery of anti-LTA antibody compositions and/or supporting materials (e.g., written instructions for using the materials, etc.) from one location to another. For example, kits include one or more enclosures (e.g., boxes) containing the relevant anti-LTA antibody compositions and/or supporting materials. As used herein, the term “fragmented kit” refers to delivery systems comprising two or more separate containers that each contain a sub-portion of the total kit components. The containers may be delivered to the intended recipient together or separately. For example, a first container may contain a composition comprising anti-LTA antibody compositions for a particular use, while a second container contains a second agent (e.g., an antibiotic or device for delivery (e.g., syringe)). Indeed, any delivery system comprising two or more separate containers that each contains a sub-portion of the total kit components are included in the term “fragmented kit.” In contrast, a “combined kit” refers to a delivery system containing all of the components of an anti-LTA antibody composition needed for a particular use in a single container (e.g., in a single box housing each of the desired components). The term “kit” includes both fragmented and combined kits.

### DETAILED DESCRIPTION OF THE INVENTION

The invention is directed to compositions and methods for treating (e.g., prophylactically and/or therapeutically) infection in a preterm infant. In particular, the invention provides anti-lipoteichoic acid (LTA) antibody compositions and methods of administering the same to a preterm infant under conditions to establish anti-LTA antibody serum concentrations effective to kill and/or prevent growth of bacteria (e.g., Staphylococci).

Compositions and methods of the invention find use in, among other things, clinical (e.g. therapeutic and preventative medicine) and research applications.

A large body of literature exists studying the protective or therapeutic effects of antibodies. Much of the early literature is focused on studying the protective effects induced by vaccine stimulated antibodies in 2 to 3 month old babies. There are over 10 different pediatric vaccines and in all cases serum concentrations of 25 μg/ml or less provide protection against bacterial disease. This suggests that relatively low concentrations of antibody can protect against infectious diseases even in hosts that are immunologically immature. In other studies using intravenous immune globulin (IVIG) for passive protection, efforts were not made to measure the specific antibody concentration present in the polyclonal immunoglobulin that was specific for the organisms being studied. Therefore the best estimate that was used to direct the clinician regarding amounts to be infused was to achieve concentrations of immunoglobulin that was found in the serum of term infants which was 7 mg/ml. This amount of polyclonal immunoglobulin represents antibodies to thousands of different epitopes present on infectious organisms, toxins, apoptotic cells and other immunogens and thus the amount of immunoglobulin specific for any epitope would be predicted to be only in the order of fractions of a microgram per ml. The fact that polyclonal immunoglobulin is not concentrated for any particular epitope explains why infusion of IVIG into preterm neonates has been unsuccessful in many major clinical trials in preventing or ameliorating infections.

Recently a pivotal trial testing the efficacy of Veronate, a polyclonal IVIG with reportedly high titers of antibody activity for a specific determinant on S. aureus failed to meet the primary endpoints of preventing staphylococcal infections. In the published report of this study, no mention was made of the antibody concentrations to the staphylococcal epitopes in these pools of polyclonal IVIG. From earlier published literature examining the opsonic activity of 100 different lots of pooled IVIG one could extrapolate that the serum antibody concentrations to the staphylococcal determinant in neonates infused with this immune globulin would be less that 10 μg/ml. Thus, the research and clinical literature have not defined a method to determine the threshold protective serum level of infused antibody to passively protect against staphylococcus or other bacteria in neonates (e.g., preterm infants (e.g., low birth weight infants (e.g., very low birth weight infants))).

Accordingly, experiments were conducted during development of embodiments of the invention in order to identify and characterize the level of serum antibody required to protect infants against staphylococcus or other infectious bacterial organisms. For example, in some embodiments, the invention provides methods for determining a dosing regimen that provides benefit (e.g., prophylactic and/or therapeutic benefit) to infants (e.g., treats and/or inhibits growth of staphylococci and/or other bacteria in the infant). In some embodiments, the invention provides in vitro models and methods to determine the level of antibody necessary to kill staphylococci or other bacteria using an opsonic assay in which the complement activity and mediator cells approximate the activity of complement and the immature effector cells present in neonates (e.g., preterm infants (e.g., low birth weight infants (e.g., very low birth weight infants (see, e.g., Example 1)))). The invention also provides anti-LTA compositions and methods that provide opsonophagocytic activity in neonates (e.g., preterm infants (e.g., low birth weight infants (e.g., very low birth weight infants))) and characterizes their activity and tolerability. (e.g., see, e.g., Examples 2 and 3). The invention provides for the first time insight into and characterization of the concentrations of serum antibody that are needed to mediate bacterial killing in an environment such as the preterm neonate where complement activity is significantly diminished and/or absent (e.g., bacterial killing occurs in the absence of complement activity). For example, in some embodiments, the invention provides novel dosing regimens that are effective at eradicating and or preventing infection (e.g., staphylococcal infection and non-staphylococcal infection) in neonates (e.g., preterm infants (e.g., low birth weight infants (e.g., very low birth weight infants))).

Any neonate or infant (e.g., preterm infant (e.g., low birth weight neonate)) at risk of sepsis or experiencing or showing the signs or symptoms of sepsis or infection (e.g., Staphylococcal infection) would benefit from the compositions (e.g., anti-LTA binding molecule composition and/or anti-LTA antibody composition) and methods of the invention. However, low birth weight preterm infants are preferred candidates.

The neonate or infant may receive anti-LTA binding molecule composition and/or anti-LTA antibody composition therapy before showing any signs or symptoms of infection or sepsis (e.g., be administered a composition of the invention as a prophylactic) or may receive anti-LTA binding molecule composition and/or anti-LTA antibody composition as a therapeutic (e.g., to treat a known or detected infection). In some embodiments, the infant or neonate is administered anti-LTA binding molecule composition and/or anti-LTA antibody composition therapy as soon as possible after birth (e.g., within the first 24 hours after birth (e.g., to achieve a prophylactic serum concentration of anti-LTA binding molecule and/or anti-LTA antibody (e.g., prior to bacterial colonization))). In some embodiments, serum concentration of anti-LTA binding molecule and/or anti-LTA antibody is achieved and/or maintained at a desired level for a set period of time after birth (e.g., for one day, two days, three days, four days, five days, six days, seven days, two weeks, three weeks, four weeks, five weeks or more. In some embodiments, a desired serum concentration of anti-LTA binding molecule and/or anti-LTA antibody is achieved within 36 hours after birth, 48 hours after birth, within 60 hours after birth, within 72 hours after birth, within 84 hours after birth or later than 84 hours after birth. In a preferred embodiment, a desired serum concentration of anti-LTA binding molecule and/or anti-LTA antibody is achieved within 72 hours after birth.

Experiments conducted during development of embodiments of the invention identified that the serum concentration of anti-LTA binding molecule and/or anti-LTA antibody needed in order to provide protection from and/or killing of bacteria in a preterm infant (e.g., low birth weight infant (e.g., very low birth weight infant) is more than that needed in order to provide protection from and/or killing of bacteria in a full term infant (See, e.g., Examples 1-3). Thus, in some embodiments, the invention provides a method of administering an anti-LTA antibody and/or anti-LTA binding molecule to a preterm infant (e.g., low birth weight infant (e.g., very low birth weight infant) that results in a higher effective serum concentration of the anti-LTA antibody and/or anti-LTA binding molecule than that required in a full term infant. For example, in some embodiments, the dosing regimen produces infant anti-LTA antibody and/or anti-LTA binding molecule serum level (e.g., a desired serum concentration) that is ≧100 μg/ml, ≧200 μg/ml, ≧300 μg/ml, ≧350 μg/ml, ≧400 μg/ml, ≧450 μg/ml, ≧500 μg/ml, ≧550 μg/ml, ≧600 μg/ml, ≧650 μg/ml, ≧700 μg/ml, ≧750 μg/ml, ≧800 μg/ml, or higher. In some embodiments, the dosing regimen produces infant anti-LTA antibody and/or anti-LTA binding molecule serum levels that are ≧500 μg/ml. In some embodiments, infant anti-LTA antibody serum levels (e.g., of ≧500 μg/ml) are maintained for one or more (e.g., 2, 3, 4, 5, 6, 7, 8, 9, 10, 11, 12, 13, 14, 15, 16, 17, 18, 19, 20, 21, 22, 23, 24 or more) days.

The invention is not limited by the route or mode of administration of an anti-LTA antibody composition or anti-LTA binding molecule composition. For example, in some embodiments the composition is administered intravenously, intramuscularly, intraperitoneally, intradermally, subcutaneously or via another route of administration described herein. In some embodiments, anti-LTA antibody composition or anti-LTA binding molecule composition is administered via intravenous infusion. The invention is not limited to the speed at which the composition is administered. In some embodiments, the composition is administered at a rate of 0.01 ml of composition per kilogram infant weight per minute, although faster and slower rates of infusion are possible. The invention is also not limited by the type of bacteria treated (e.g., therapeutically and/or prophylactically) using a composition comprising anti-LTA antibody composition or anti-LTA binding molecule composition of the invention. For example, in some embodiments, any type of gram positive bacteria expressing LTA can be treated including, but not limited to, S. epidermidis, S. epidermidis strain Hay, S. hemolyticus, S. hominus and serotypes of S. aureus. In some embodiments, bacterial growth and/or infection (e.g., sepsis) is prevented and/or treated in infants receiving anti-LTA antibody therapy. In some embodiments, the method comprises administration of an anti-LTA antibody composition and one or more other antibacterial agents. In some embodiments, the infant resides within an intensive care unit (ICU (e.g., neonatal ICU, surgical ICU or other ICU of a hospital)). In some embodiments, an infant is administered a composition of the invention (e.g., an anti-LTA binding molecule composition or anti-LTA antibody composition) together with one or more therapies (e.g., intravenous fluids, antibiotics, blood pressure medicines, ventilation, hemodialysis, transfusion of blood products, nutrition, immunostimulants, etc.).

In some embodiments, the invention provides anti-LTA antibody composition and/or binding molecule composition comprising a pharmaceutically acceptable carrier (e.g., a pharmaceutical composition comprising an anti-LTA antibody). The present invention is not limited by the type of pharmaceutically acceptable carrier utilized. Indeed, compositions useful in a pharmaceutical formulation are well known in the art and can include any one or more of the types of carries described below. In some embodiments, a pharmaceutical composition comprising an anti-LTA antibody composition and/or binding molecule composition is administered to a subject in order to achieve and maintain a desired serum concentration of anti-LTA binding molecule and/or anti-LTA antibody in the subject. The invention also provides the use of an anti-LTA antibody composition and/or anti-LTA binding molecule composition in the manufacture of a medicament for the treatment of a disease or disorder associated with a Gram positive bacterial infection (e.g., a S. aureus bacterial infection, a S. epidermidis bacterial infection, a coagulase negative staphylococci bacterial infection, or a Streptococcus mutans bacterial infection). Thus, in some preferred embodiments, a medicament of the invention (e.g., comprising anti-lipoteichoic acid antibody composition) is administered to a subject as a therapeutic (e.g., to prevent or attenuate a disease (e.g., by providing to the subject total or partial immunity against the disease or the total or partial attenuation (e.g., suppression) of a sign, symptom or condition of the disease)).

The present invention is not limited by the type of anti-LTA antibody or anti-LTA binding molecule utilized in a anti-LTA antibody composition or anti-LTA binding molecule composition. A variety of anti-LTA antibodies and/or anti-LTA binding molecules may be used including, but not limited to, antibodies or binding molecules that bind to LTA with a binding affinity greater than (or equal to) about 10⁶ M⁻¹, 10⁷ M⁻¹, 10⁸M⁻¹, 10⁹ M, or 10¹⁰, (including affinities intermediate of these values). In a preferred embodiment, the anti-LTA antibody is a chimeric IgG1 antibody derived from a murine monoclonal antibody, 96-110 (A110). The hybridoma cell line which produces 96-110 was deposited on Jun. 13, 1997, with the ATCC according to the provisions of the Budapest Treaty and was assigned ATCC accession number HB-12368. The isolation and anti-staphylococcal properties have been described in U.S. Pat. No. 6,610,293, the entire contents of which are herein incorporated by reference. In some embodiments, the anti-LTA antibody is a chimeric IgG1 antibody derived from a murine monoclonal antibody, 96-120 (A120). Chimeric LTA antibody, A120, is described in U.S. Pat. No. 7,250,494, the entire contents of which are herein incorporated by reference. As described in U.S. Pat. Nos. 6,610,293 and 7,250,494, the A110 and A120 antibodies have been shown to bind and opsonize whole Gram positive bacteria, including multiple strains of S. epidermidis and S. aureus, thereby enhancing phagocytosis and killing of such bacteria in vitro and enhancing protection from lethal infection of such bacteria in vivo. While an understanding of a mechanism is not necessary to practice the invention and the invention is not limited to any particular mechanism of action, in some embodiments, anti-LTA binding molecules and/or anti-LTA antibodies of the invention to block binding of LTA on bacteria to epithelial cells, and hence the subsequent invasion of the bacteria, and are also opsonic, thereby enhancing clearance of the bacteria from tissues and blood. Therefore, the invention provides protection against and therapeutic treatment of infection caused by Gram positive bacteria. Thus, compositions and methods of the invention possess properties which render them useful for prevention and treatment of Staphylococcal infection in a neonatal subject (e.g., preterm infant (e.g., low birth weight infant (e.g., very low birth weight infant))). In a preferred embodiment, an anti-LTA antibody composition and/or an anti-LTA binding molecule composition is administered (e.g., infused in) to a LBW infant (e.g., preterm infant (e.g., VLBW infant)) at 100 mg anti-LTA antibody per kilogram (kg) infant weight daily for 3 days (e.g., days 0, 1, and 2) followed by administration (e.g., infusion) of 100 mg anti-LTA antibody per kg infant weight at weeks 1, 2, and 3 thereafter (e.g., on days 9, 16 and 23).

Anti-LTA antibodies and/or anti-LTA binding molecules can be prepared as pharmaceutical formulations according to any method known to the art for the manufacture of pharmaceuticals, as described below. Such drugs may contain coloring agents and/or preserving agents. Any anti-LTA antibody and/or anti-LTA binding molecule can be admixed with nontoxic pharmaceutically acceptable excipients which are suitable for manufacture.

In general, anti-LTA antibody compositions and/or anti-LTA binding molecule compositions may be administered as pharmaceutical compositions by any method known in the art for administering therapeutic drugs. However, the compositions used in the methods of the invention are preferably administered via intravenous infusion (e.g., as described in Example 2).

Single or multiple administrations of a composition can be administered depending on the frequency, amount of dosage, and half life of the composition. When practicing the methods of the invention, a number of general laboratory tests can be used to assist in the diagnosis, progress and prognosis of a low birth weight preterm infant at risk for infection, including monitoring of parameters such as blood counts, temperature, stool samples, etc. These procedures can be helpful because all patients metabolize and react to drugs uniquely. Different patients may require different dosage regimens and formulations.

The LTA antibodies of the present invention may be used in the prevention and/or treatment of infection caused by Gram positive bacteria, such as coagulase positive and coagulase negative staphylococci, in humans or animals. In particular, the antibodies of the invention may be used in the prevention and/or treatment of sepsis caused by Gram positive bacteria, such as coagulase positive and coagulase negative staphylococci. More particularly, the antibodies of the intention may be used in the prevention of Staphylococcal infections, sepsis, bacteremia, and inflammation in low birth weight neonates, including very low birth weight and extremely low birth weight neonates, e.g., birth weight between 600 and 1300 grams.

In some embodiments, compositions in accordance with the invention may be used for the specific detection of staphylococci, for the prevention of infection from staph bacteria, for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies to LTA, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. In the present case, optimized chimeric antibodies to LTA have been generated and isolated and shown to have high binding affinity to several strains of live staphylococcal bacteria.

As described herein, in some embodiments, the invention is directed inter alia to prevention or treatment of infection caused by Gram positive bacteria by administration of antibodies which bind LTA. Preferably, the present invention is directed to the prevention or treatment of Gram positive bacteria in neonates. The invention is also directed to use of the disclosed LTA antibodies in the manufacture of a medicament for the treatment or prevention of infection caused by Gram positive bacteria. Preferably, the invention is directed to the use of the disclosed LTA antibodies in the manufacture of a medicament for the treatment or prevention of infection caused by Gram positive bacteria in neonates. In one aspect, the invention provides methods of preventing or treating infection caused by Gram positive bacteria. Such Gram positive bacteria include both coagulase positive and coagulase negative Staphylococci. Some methods of the invention comprise administering an effective dosage of an antibody that specifically binds to LTA to the patient. Such methods are particularly useful for preventing or treating infection caused by Gram positive bacteria in human patients. Therapeutic antibodies of the invention are typically substantially pure from undesired contaminant. This means that an antibody is typically at least about 50% w/w (weight/weight) pure, as well as being substantially free from interfering proteins and contaminants. Sometimes the antibodies are at least about 80% w/w and, more preferably at least 90 or about 95% w/w pure. However, using conventional protein purification techniques, homogeneous peptides of at least 99% w/w pure can be obtained.

The methods can be used on both asymptomatic patients and those currently showing symptoms of infection. The antibodies used in such methods can be human, humanized, chimeric or nonhuman antibodies, or fragments thereof (e.g., antigen binding fragments) and can be monoclonal or polyclonal, as described herein. In another aspect, the invention features administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

In prophylactic applications, pharmaceutical compositions or medicaments are administered to a patient susceptible to, or otherwise at risk of, an infection caused by Gram positive bacteria in an amount sufficient (e.g., as described herein, see, e.g., Examples 1-3) to eliminate or reduce the risk, lessen the severity, or delay the outset of the infection. In therapeutic applications, compositions or medicaments are administered to a patient suspected of, or already suffering from such infection caused by Gram positive bacteria in an amount sufficient to cure, or at least partially arrest, the symptoms of the infection. An amount adequate to accomplish therapeutic or prophylactic treatment is defined as a therapeutically- or prophylactically-effective dose. In both prophylactic and therapeutic regimes, antibodies are usually administered in several dosages until a sufficient immune response has been achieved.

Subjects can be administered such doses hourly, daily, on alternative days, weekly or according to any other schedule determined by empirical analysis. The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance, i.e., provide at least some measure of prevention of infection caused by Gram positive bacteria. Such an amount is defined to be a “prophylactic effective dose.” Such prophylactic therapy as described herein may be primary or supplemental to additional treatment, such as antibiotic therapy, for an infection caused by Gram positive bacteria, an infection caused by a different agent, or an unrelated disease.

Therapeutic antibodies can be administered by parenteral, topical, intravenous, oral, subcutaneous, intraarterial, intracranial, intraperitoneal, intranasal or intramuscular means for prophylactic and/or therapeutic treatment. The most typical route of administration of the antibodies of the invention is intravenous although other routes can be equally effective. For example, the antibodies of the invention can also be administered subcutaneously or via intramuscular injection. Intramuscular injection is most typically performed in the arm or leg muscles. Intramuscular injection or intravenous infusion are preferred for administration of antibody. In some methods, antibodies are administered as a sustained release composition or device, such as a MEDIPAD™ device.

Antibodies of the invention can optionally be administered in combination with other agents that are at least partly effective in treatment of staphylococcal infections, e.g., antibiotics and other anti-bacterial agents. In certain embodiments, an LTA antibody of the invention is administered in combination with a second immunogenic or immunologic agent. For example, an LTA antibody of the invention can be administered in combination with another antibody to LTA. In other embodiments, an LTA antibody of the invention is administered to a patient who has received or is receiving an LTA vaccine. Agents of the invention can also be administered in combination with other agents that enhance access of the therapeutic agent to a target cell or tissue, for example, liposomes and the like. Co-administering such agents can decrease the dosage of a therapeutic antibody or antigen-binding fragment needed to achieve a desired effect.

Antibodies of the invention are often administered as pharmaceutical compositions comprising an active therapeutic antibody and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)). The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%70%. Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin patch or using transferosomes (Paul et al., Eur. J. Immunol. 25:3521 (1995); Cevc et al., Biochem. 15 Biophys. Acta 1368:201-15 (1998)).

In some embodiments, a composition of the present invention may be formulated for administration by any route, such as mucosal, oral, topical, intravenous, subcutaneous, parenteral or other route described herein. The compositions may be in any one or more different forms including, but not limited to, tablets, capsules, powders, granules, lozenges, foams, creams or liquid preparations.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the anti-LTA antibody and/or anti-LTA binding molecule of the formulation. In some embodiments, compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising an anti-LTA binding molecule and/or anti-LTA antibody is co-administered with one or more antibiotics. For example, one or more antibiotics may be administered with, before and/or after administration of an anti-LTA binding molecule/anti-LTA antibody. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines. In some embodiments, the antibiotic agent used is any antibiotic shown to have antimicrobial activity (e.g., anti-Staphylococcal activity). In some embodiments, the antibiotic is an anti-staphylococcal antibiotic (e.g., characterized as having anti-staphylococcal activity).

There are an enormous amount of antimicrobial agents currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

In some embodiments, the invention provides compositions and methods for the co-administration of a composition comprising an anti-LTA binding molecule/anti-LTA antibody together with an antibiotic to a subject (e.g., an adult or an infant (e.g., preterm infant (e.g., low birth weight preterm infant))). For example, in some embodiments, an anti-LTA binding molecule and/or anti-LTA antibody is co-administered with one or more antibiotics under conditions such that an anti-LTA antibody and/or anti-LTA binding molecule serum level of ≧100 μg/ml, ≧200 μg/ml, ≧300 μg/ml, ≧350 μg/ml, ≧400 μg/ml, ≧450 μg/ml, ≧500 μg/ml, ≧550 μg/ml, ≧600 μg/ml, ≧650 μg/ml, ≧700 μg/ml, ≧750 μg/ml, ≧800 μg/ml, ≧900 μg/ml, ≧1 mg/ml, ≧1.2 mg/ml, ≧1.4 mg/ml, ≧1.5 mg/ml, ≧1.75 mg/ml, ≧2.0 mg/ml, or higher is achieved, together with an antibiotic serum level of ≧0.5 μg/ml, ≧0.75 μg/ml, ≧1.0 μg/ml, ≧1.5 μg/ml, ≧2.0 μg/ml, ≧3.0 μg/ml, ≧4.0 μg/ml, ≧5.0 μg/ml, ≧6.0 μg/ml, ≧6.5 μg/ml, ≧7.0 μg/ml, ≧7.5 μg/ml, ≧10 μg/ml, ≧15 μg/ml, ≧30 μg/ml, ≧60 μg/ml, ≧100 μg/ml, ≧200 μg/ml, ≧300 μg/ml, ≧500 μg/ml, ≧750 μg/ml, ≧1 mg/ml, ≧1.5 mg/ml, ≧2.0 mg/ml in the subject. In some embodiments, co-administration of an anti-LTA binding molecule and/or anti-LTA antibody with an antibiotic produces a serum level of ≧300 μg/ml anti-LTA binding molecule and/or anti-LTA antibody and ≧4.0 μg/ml antibiotic. In some embodiments, co-administration of an anti-LTA binding molecule and/or anti-LTA antibody with an antibiotic produces a serum level of ≧350 μg/ml anti-LTA binding molecule and/or anti-LTA antibody and ≧4.5 μg/ml antibiotic. In some embodiments, co-administration of an anti-LTA binding molecule and/or anti-LTA antibody with an antibiotic produces a serum level of ≧400 μg/ml anti-LTA binding molecule and/or anti-LTA antibody and ≧5.0 μg/ml antibiotic. In some embodiments, co-administration of an anti-LTA binding molecule and/or anti-LTA antibody with an antibiotic produces a serum level of ≧450 μg/ml anti-LTA binding molecule and/or anti-LTA antibody and ≧5.0 μg/ml antibiotic. In some embodiments, co-administration of an anti-LTA binding molecule and/or anti-LTA antibody with an antibiotic produces a serum level of ≧500 μg/ml anti-LTA binding molecule and/or anti-LTA antibody and ≧5.0 μg/ml antibiotic. In some embodiments, co-administration of an anti-LTA binding molecule and/or anti-LTA antibody with an antibiotic produces a serum level of ≧550 μg/ml anti-LTA binding molecule and/or anti-LTA antibody and ≧5.0 μg/ml antibiotic. In some embodiments, anti-LTA antibody serum levels (e.g., of ≧300 μg/ml, ≧350 μg/ml, ≧400 μg/ml, ≧450 μg/ml, ≧500 μg/ml or higher) and antibiotic levels (e.g., ≧1.0 μg/ml, ≧1.5 μg/ml, ≧2.0 μg/ml, ≧3.0 μg/ml, ≧4.0 μg/ml, ≧5.0 μg/ml) are maintained for one or more (e.g., 2, 3, 4, 5, 6, 7, 8, 9, 10, 11, 12, 13, 14, 15, 16, 17, 18, 19, 20, 21, 22, 23, 24 or more) days. In some embodiments, anti-LTA antibody serum levels and antibiotic levels are maintained for three days. In some embodiments, anti-LTA antibody serum levels and antibiotic levels are maintained for four days. In some embodiments, anti-LTA antibody serum levels and antibiotic levels are maintained for five days. In some embodiments, anti-LTA antibody serum levels and antibiotic levels are maintained for six days. In some embodiments, anti-LTA antibody serum levels and antibiotic levels are maintained for seven days. The invention is not limited by the type of antibiotic co-administered with an anti-LTA binding molecule and/or anti-LTA antibody. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines. In some embodiments, the antibiotic co-administered with an anti-LTA binding molecule and/or anti-LTA antibody is a penicillin. In some embodiments, the antibiotic co-administered with an anti-LTA binding molecule and/or anti-LTA antibody is vancomycin. In some embodiments, the antibiotic co-administered with an anti-LTA binding molecule and/or anti-LTA antibody is a β-lactam antibiotic. In some embodiments, the antibiotic agent is any antibiotic shown to have antibacterial activity (e.g., anti-Staphylococcal activity). In some embodiments, the antibiotic is an anti-staphylococcal antibiotic (e.g., characterized as having anti-staphylococcal activity).

Accordingly, in some embodiments, the invention provides the use of a composition comprising an anti-LTA binding molecule and/or anti-LTA antibody and an antibiotic in the manufacture of a medicament for the treatment or prevention of Staphylococcal infection in a subject (e.g., an adult or infant (e.g., preterm infant (e.g., low birth weight preterm infant))). In some embodiments, the medicament is formulated for administration to a subject (e.g., an adult or infant) under conditions such that the serum concentration of the anti-LTA binding molecule and/or anti-LTA antibody reaches a level described above. In some embodiments, the medicament is formulated for administration to a subject (e.g., an adult or infant) under conditions such that the serum concentration of one or more antibodies reach a level described above. In some embodiments, the medicament is a composition comprising both an anti-LTA binding molecule and/or anti-LTA antibody and one or more antibiotics. In some embodiments, the medicament is formulated for administration to a subject under conditions such that about 1-100 mg/kg/dose of antibiotic and about 1-500/mg/kg anti-LTA antibody and/or an anti-LTA binding molecule are administered to the subject. In some embodiments, the medicament is formulated for administration to a subject under conditions such that about 1-50 mg/kg/dose of antibiotic and about 5-250/mg/kg anti-LTA antibody and/or an anti-LTA binding molecule are administered to the subject. In some embodiments, the medicament is formulated for administration to a subject under conditions such that about 2-20 mg/kg/dose of antibiotic and about 50-100/mg/kg anti-LTA antibody and/or an anti-LTA binding molecule are administered to the subject. In some embodiments, the medicament is formulated for administration to a subject under conditions such that about 5-15 mg/kg/dose of antibiotic and about 75-100/mg/kg anti-LTA antibody and/or an anti-LTA binding molecule are administered to the subject. In some embodiments, the medicament is formulated for administration to a subject under conditions such that about 10-15 mg/kg/dose of antibiotic and about 85-100/mg/kg anti-LTA antibody and/or an anti-LTA binding molecule are administered to the subject. In some embodiments, the medicament is formulated for administration to a subject (e.g., an adult or infant) under conditions such that about 15 mg/kg/dose of antibiotic and about 100/mg/kg anti-LTA antibody and/or an anti-LTA binding molecule are administered to the subject. In some embodiments, the medicament is formulated for administration to a subject one or more (e.g., two, three, four or more) times per day.

The invention also provides methods of treating a subject comprising providing a subject and a composition comprising an anti-LTA binding molecule/anti-LTA antibody and a composition comprising one or more antibiotics and co-administering the compositions to the subject. The composition comprising an anti-LTA binding molecule and/or anti-LTA antibody may be combined with a composition comprising one or more antibiotics (e.g., and the single composition administered to a subject). Alternatively, the composition comprising an anti-LTA binding molecule and/or anti-LTA antibody may be separate from the composition comprising one or more antibiotics, and the two compositions co-administered (e.g., the two compositions are administered at the same time, or, one composition is administered and then the second administered). Those of skill in the art understand that the formulations and/or routes of administration of the compositions used may vary. Likewise, an appropriate dosage for co-administration can be readily determined by one skilled in the art. In some embodiments, co-administration of a composition comprising an anti-LTA binding molecule/anti-LTA antibody and one or more antibiotics to a subject prevents signs and/or symptoms of infection (e.g., caused by pathogenic bacteria) in the subject. In a preferred embodiment, co-administration of an anti-LTA binding molecule/anti-LTA antibody and one or more antibiotics to a subject allows the subject to survive a lethal exposure to a pathogenic bacteria (e.g., pathogenic Staphylococcal aureus (e.g., antibiotic resistant Staphylococcal aureus (e.g., vancomycin resistant Staphylococcal aureus, methicillin resistant Staphylococcal aureus, etc.))).

In some embodiments, co-administration of an anti-LTA binding molecule/anti-LTA antibody and one or more antibiotics is utilized as a prophylactic treatment (e.g., to prevent infection (e.g., caused by pathogenic bacteria)). In some embodiments, co-administration of an anti-LTA binding molecule/anti-LTA antibody and one or more antibiotics is utilized as a therapeutic treatment (e.g., to treat bacterial infection (e.g., caused by pathogenic bacteria (e.g., Staphylococcal infection (e.g., antibiotic resistant Staphylococcus aureus)))). For example, in some embodiments, co-administration of an anti-LTA binding molecule/anti-LTA antibody and one or more antibiotics to a subject occurs within an hour of exposure to pathogenic bacteria (e.g., capable of causing infection (e.g., sepsis)). In some embodiments, co-administration occurs within 2 hours, 4 hours, 6 hours, 9 hours, 12 hours, 18 hours, 24 hours, 36 hours, 48 hours, 60 hours, 72 hours, or more of exposure of the subject to a pathogenic bacteria (e.g., that causes infection in the subject). In some embodiments, co-administration of an anti-LTA binding molecule/anti-LTA antibody and one or more antibiotics occurs after infection is identified in a subject. Under such circumstances, an anti-LTA binding molecule/anti-LTA antibody and one or more antibiotics are co-administered as soon as possible after the infection is identified (e.g., within minutes or hours). In some embodiments, co-administration of an anti-LTA binding molecule/anti-LTA antibody and one or more antibiotics to a subject increases the likelihood of survival of the subject from infection compared to the likelihood of survival of the subject from infection if the subject were administered one or more antibiotics in the absence of anti-LTA binding molecule/anti-LTA antibody. In some embodiments, the rate of survival of a subject from infection is increased about 10%, 20%, 30%, 40%, 50%, 60%, 70%, 80%, 90% or more when an anti-LTA binding molecule/anti-LTA antibody is co-administered with one or more antibiotics compared to the survival rate of a subject when one or more antibiotics is administered in the absence of anti-LTA binding molecule/anti-LTA antibody to the subject.

The present invention also includes methods involving co-administration of a composition comprising an anti-LTA binding molecule and/or anti-LTA antibody with one or more additional active and/or immunostimulatory agents (e.g., an antibiotic, anti-oxidant, etc.). Indeed, it is a further aspect of this invention to provide methods for enhancing prior art anti-microbial methods (e.g., antibacterial methods) and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, adjuvants, etc.) can be any of the well-known agents in the art, including, but not limited to, those that are currently in clinical use.

In some embodiments, a composition comprising an anti-LTA binding molecule and/or anti-LTA antibody is administered to a subject via more than one route. For example, a subject may benefit from receiving intravenous infusion and, additionally, receiving one or more other routes of administration (e.g., parenteral, intrathecal, intraarterial, etc.). Thus, although an understanding of the mechanism is not necessary to practice the present invention and the present invention is not limited to any particular mechanism of action, in some embodiments, it is contemplated that a subject administered a composition of the present invention via multiple routes of administration may have a stronger antibacterial response or protection than a subject administered a composition via just one route.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In some embodiments, a composition comprising an anti-LTA binding molecule and/or anti-LTA antibody is formulated in a concentrated dose that can be diluted prior to administration to a subject. For example, dilutions of a concentrated composition may be administered to a subject such that the subject receives any one or more of the specific dosages provided herein.

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Thus, it is not intended that the present invention be limited to any particular subject and/or application setting.

A composition comprising an anti-LTA binding molecule and/or anti-LTA antibody finds use where the nature of the infectious and/or disease causing agent is known, as well as where the nature of the infectious and/or disease causing agent is unknown.

In some embodiments, the present invention provides a kit comprising a composition comprising an anti-LTA binding molecule and/or anti-LTA antibody. In some embodiments, the kit further provides a device for administering the composition. The present invention is not limited by the type of device included in the kit. In some embodiments, a kit comprises a composition comprising an anti-LTA binding molecule and/or anti-LTA antibody in a concentrated form (e.g., that can be diluted prior to administration to a subject).

In some embodiments, all kit components are present within a single container (e.g., vial or tube). In some embodiments, each kit component is located in a single container (e.g., vial or tube). In some embodiments, one or more kit component are located in a single container (e.g., vial or tube) with other components of the same kit being located in a separate container (e.g., vial or tube). In some embodiments, a kit comprises a buffer. In some embodiments, the kit further comprises written material comprising instructions for using the composition (e.g., providing instructions for dosing). Thus, in some embodiments, a kit contains labeling providing directions for use of the kit. The labeling may also include a chart or other correspondence regime correlating levels of measured label with levels of LTA antibodies. The term labeling refers to any written or recorded material that is attached to, or otherwise accompanies a kit at any time during its manufacture, transport, sale or use. For example, the term labeling encompasses advertising leaflets and brochures, packaging materials, instructions, audio or videocassettes, computer discs, as well as writing imprinted directly on kits.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. See, for example, Molecular Cloning: A Laboratory Manual, 2nd edition. (Sambrook, Fritsch and Maniatis, eds.), Cold Spring Harbor Laboratory Press, 1989; DNA Cloning, Volumes I and II (D. N. Glover, ed), 1985; Oligonucleotide Synthesis, (M. J. Gait, ed.), 1984; U.S. Pat. No. 4,683,195 (Mullis et al.); Nucleic Acid Hybridization (B. D. Hames and S. J. Higgins, eds.), 1984; Transcription and Translation (B. D. Hames and S. J. Higgins, eds.), 1984; Culture of Animal Cells (R. I. Freshney, ed). Alan R. Liss, Inc., 1987; Immobilized Cells and Enzymes, IRL Press, 1986; A Practical Guide to Molecular Cloning (B. Perbal), 1984; Methods in Enzymology, Volumes 154 and 155 (Wu et al., eds), Academic Press, New York; Gene Transfer Vectors for Mammalian Cells (J. H. Miller and M. P. Calos, eds.), 1987, Cold Spring Harbor Laboratory; Immunochemical Methods in Cell and Molecular Biology (Mayer and Walker, eds.), Academic Press, London, 1987; Handbook of Experiment Immunology, Volumes I-IV (D. M. Weir and c.c. Blackwell, eds.), 1986; Manipulating the Mouse Embryo, Cold Spring Harbor Laboratory Press, 1986.

### EXAMPLES

The following examples serve to illustrate certain preferred embodiments and aspects of the present invention and are not to be construed as limiting the scope thereof.

In the experimental disclosure which follows, the following abbreviations apply: eq (equivalents); μ(micron); M (Molar); μM (micromolar); mM (millimolar); N (Normal); mol (moles); mmol (millimoles); pmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar); ° C. (degrees Centigrade); and PBS (phosphate buffered saline).

### Example 1

### In Vitro and Animal Models to Characterize Prevention of Staphylococcal Infections in Neonates, Low Birth Weight (LBW) and Very Low Birth Weight (VLBW) Infants Using Monoclonal Anti-Lipoteichoic Acid (LTA) Antibody

There are a number of unique features in the immune and inflammatory responses of premature infants that have led to the failure of the majority of clinical trials that have utilized intravenous immune globulin (IVIG) to prevent infectious diseases in preterm and/or low birth weight infants (e.g., VLBW infants). The ability of IVIG to mediate bacterial clearance is dependent on many different cellular and blood borne components each of which contributes at some level to the effectiveness of the antibody in mediating bacterial killing. At the cellular level, the host needs to provide phagocytic cells that are capable of migrating to the site of bacterial entry, bind to the organism via many different surface receptors, engulf and opsonize the bacteria and finally through the cell's various enzymatic compartments mediate bacterial lysis. These cells are comprised predominantly of neutrophils but include macrophages as well. At the blood borne level there are opsonins, the complement system, collectins and other molecules that facilitate binding of the bacteria to the phagocytic cells, their opsonization and lysis. In VLBW infants all of these components are reduced both in number and in activity and thus provide a very compromised effector system that cannot interact optimally with infused immunoglobulin. Thus, in an effort to reflect the conditions that would support the ability of an anti-LTA antibody composition (e.g., PAGIBAXIMAB, described herein) to protect VLBW infants against staphylococcal infection in this compromised environment, in vitro assays and animal models were developed that mimic the compromised neonatal effector system. These models were utilized to study and characterize the anti-staphylococcal activity of anti-LTA antibody compositions. These assays were important in directing the selection of an appropriate serum concentration of antibody that would be protective in preterm neonates and was markedly different from concentrations that have been thought to be protective based on data from other antibody and vaccine trials.

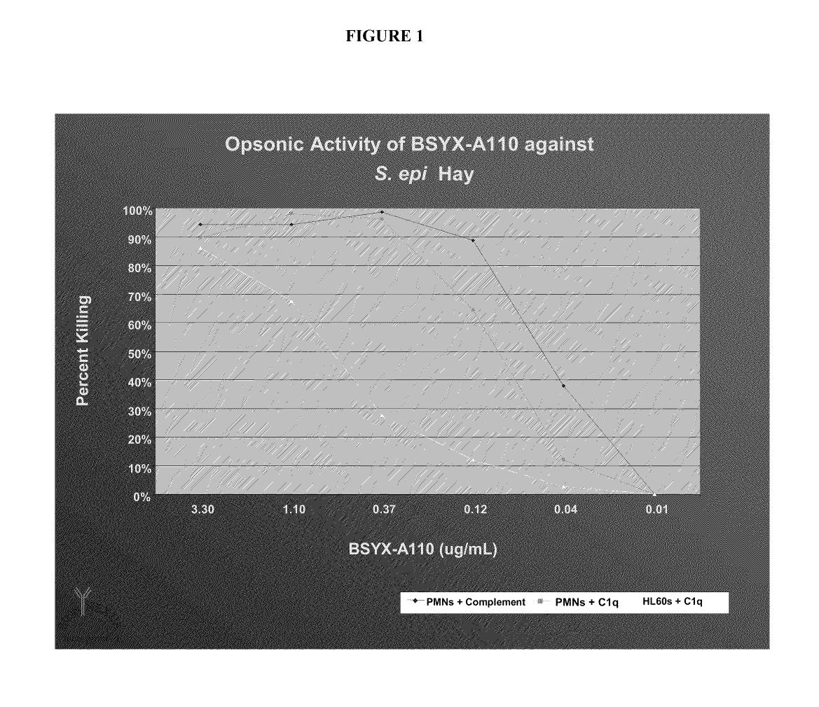

In vitro assays. The cellular based in vitro assay that is regarded as the most accurate surrogate for predicting antibody efficacy in clearing bacteria from the circulation is the opsonophagocidal assay (opsonic assay). The U.S. Food and Drug Administration (FDA) currently accepts this assay as a predictor of vaccine efficacy in place of conducting full clinical trials measuring reduction of disease incidence. The standard opsonic assay uses neutrophils or whole human blood as the source of phagocytic cells and uses human serum or rabbit complement as the source of complement and other opsonins. Using this standard opsonic assay, an anti-LTA antibody composition (PAGIBAXIMAB) mediated bacterial killing at doses as low as 0.2 μg/ml. The revised opsonic assay developed uses cells from a poorly differentiated myelo-monocytic cell line (HL-60) as the source of phagocytic cells and C1q as the sole source of complement with no further addition of any other serum derived opsonins. Using this assay the activity of the anti-LTA antibody composition (PAGIBAXIMAB) was approximately 10 fold lower than in the standard opsonic assay. Thus, the invention provides that anti-LTA antibody composition (PAGIBAXIMAB) is an effective opsonic antibody but that greater serum concentrations of antibody were required with VLBW neonates where the cellular components and the serum components mediating opsonization are compromised as compared to the term infant or the adult (See FIG. 1).

In evaluating 26 different clinical isolates using the HL-60 dependent opsonic assay it was determined that a wide range of concentrations of anti-LTA antibody composition existed that could mediate bacterial killing but that to insure killing of greater than 90% of the isolates a concentration of 400 μg/ml was required.

Animal models. An animal model was designed that would closely mimic the environment of the VLBW neonate. Suckling 2 day old rats were injected with intralipids to suppress their phagocytic system and a subcutaneous catheter was placed to mimic the catheters that are placed in VLBW neonates. The ability of anti-LTA antibody composition (PAGIBAXIMAB) was characterized for the ability to protect the suckling rats from mortality induced by S. aureus or S. epidermidis. In both cases it was observed that optimal protection required dosing that would achieve a serum concentration of at least 400 μg/ml in the suckling rats.

### Example 2

### Anti-Staphylococcal Monoclonal Antibody Prevents Staphylococcal Bloodstream Infections in Very-Low-Birth-Weight Neonates

Very-low-birth-weight (VLBW) neonates (<1,500-g birth weight) are at high risk for late-onset (>72 h of life) hospital-acquired sepsis (Fanaroff et al. 1998. Pediatr. Infect. Dis. J. 17:593-598., Gladstone et al. 1990. Pediatr. Infect. Dis. J. 9:819-825., Gray et al. 1995. Pediatrics 95:225-230., herein incorporated by reference in their entireties). Such infections are a major cause of morbidity, prolong time in the hospital and intensive care unit, increase the need for antibiotics, and further increase the substantial cost of medical care for these infants (Brodie et al. 2000. Pediatr. Infect. Dis. J. 19:56-62., Gray et al. 1995. Pediatrics 95:225-230., herein incorporated by reference in their entireties). Staphylococci, including coagulase-negative staphylococci (CONS) and Staphylococcus aureus, are responsible for between 56 and >75% of hospital-acquired, late-onset neonatal sepsis (Fanaroff et al. 1998. Pediatr. Infect. Dis. J. 17:593-598., Stoll et al. 2002. Pediatrics 110:285-291., herein incorporated by reference in their entireties). Recent reports show continuing increases in resistance of staphylococci to antimicrobial agents (Eady & Cove. 2003. Curr. Opin. Infect. Dis. 16:103-124., Smith et al. 1999. N. Engl. J. Med. 340:493-501., Srinivasan et al. 2002. Clin. Microbiol. Rev. 15:430-438., herein incorporated by reference in their entireties). Frequent and prolonged exposures to antimicrobials have been demonstrated to increase the risk of developing infections with resistant organisms (Srinivasan et al. 2002. Clin. Microbiol. Rev. 15:430-438., Tegnell. 2003. Microb. Drug Resist. 9:1-6., herein incorporated by reference in their entireties). Therapeutic products and strategies that could prevent infections would minimize the need for antimicrobial products (Jones. 1996. Diagn. Microbiol. Infect. Dis. 25:153-161., herein incorporated by reference in its entirety).

Lipoteichoic acid (LTA) is a highly conserved epitope in the staphylococcal cell wall that inhibits phagocytosis of bacteria in vitro, induces the cytokine cascade through stimulation of Tolllike receptors, and may be necessary for staphylococcal survival (trundling & Schneewind. 2007. Proc. Natl. Acad. Sci. USA 104:8478-8483., Hermann et al. 2002. Eur. J. Immunol. 32:541-551., Neuhaus & Baddiley. 2003. Microbiol. Mol. Biol. Rev. 67:686-723., Raynor et al. 1981. Clin. Immunol. Immunopathol. 19:181-189., herein incorporated by reference in their entireties). An anti-LTA murine/human chimeric monoclonal antibody, PAGIBAXIMAB, was developed by recombinant DNA technology, and its activity was confirmed in vitro and in animal studies against CONS (Weisman et al. 2009. Immunopharmacol. 9:639-644., herein incorporated by reference in its entirety) and S. aureus. 500 μg/ml was determined to be the protective level of PAGIBAXIMAB, on the basis of preclinical PAGIBAXIMAB bactericidal activity against a number of clinical isolates in vitro and in staphylococcal sepsis models in suckling animals. PAGIBAXIMAB resistance bound 24 different strains of CONS and S. aureus and demonstrated increased bacterial killing in vitro against all of these strains. There was a clear dose-response curve with 400 μg/ml required to show the maximum killing activity on all of the strains tested and lower doses being less bactericidal. In a suckling rat model of sepsis caused by CONS, PAGIBAXIMAB significantly increased survival at a dose of 80 mg/kg of body weight, and the effect of 40 mg/kg was significantly lower. This was associated with suckling rat serum PAGIBAXIMAB concentrations of approximately 275 to 400 μg/ml. In a lethal suckling rat model of S. aureus sepsis, PAGIBAXIMAB significantly increased survival at 80 mg/kg/dose, and protection was lower at doses of 40 mg/kg. This was associated with suckling rat serum PAGIBAXIMAB concentrations of 400 to 500 μg/ml. In view of the fact that VLBW infants have compromised innate immunity, excess antibody was administered to ensure bactericidal activity under conditions in which the effector system might be compromised as occurs in the VLBW infant. 500 μg/ml of antibody was selected as the protective level. PAGIBAXIMAB is safe and tolerable in healthy human adults as a single intravenous (i.v.) dose at 3 or 10 mg per kilogram (Weisman et al. 2009. Immunopharmacol. 9:639-644., herein incorporated by reference in its entirety).

Study design. A phase 1/2, randomized, double-blind, placebo-controlled, dose escalation study assessing the safety and pharmacokinetic profile of four dose levels of PAGIBAXIMAB was performed. Based on previous studies of a neonatal monoclonal antibody to prevent infection (Subramanian et al. 1998. Pediatr. Infect. Dis. J. 17:110-115., herein incorporated by reference in its entirety), monoclonal antibodies to treat infection (Angus et al. 2000. JAMA 283:1723-1730., Derkx et al. 1999. Clin. Infect. Dis. 28:770-777., herein incorporated by reference in their entireties), PAGIBAXIMAB in animal models (Weisman et al. 2001. Pediatr. Res. 49:301A., herein incorporated by reference in its entirety), neonatal suckling rat toxicity studies, and a PAGIBAXIMAB study of adults (Weisman et al. 2009. Int. Immunopharmacol. 9:639-644., herein incorporated by reference in its entirety), the four dose levels of PAGIBAXIMAB chosen for the present study were 10, 30, 60, and 90 mg/kg. Based on these in vitro and animal studies, serum PAGIBAXIMAB levels of 500 μg/ml were anticipated to provide protection against the broadest spectrum of CONS and S. aureus sepsis in VLBW neonates.

Study entry criteria. Eligible patients were infants with a birth weight of 700 to 1,300 g, 3 to 7 days of age (inclusive), inpatients in the neonatal intensive care unit with i.v. access, and expected to live at least 1 week following infusion. Patients with any of the following conditions were excluded from eligibility: clinically overt systemic infection; life-threatening hemodynamic instability; severe congenital anomaly or genetic disorder; known or suspected hepatic or renal insufficiency; persistent seizure disorder; immunodeficiency due to reasons other than prematurity; a history of immune globulin administration prior to first study drug infusion; any history (patient or mother) of a hypersensitivity or severe vasomotor reaction to immunoglobulin G (IgG) or blood products; abnormal laboratory findings, including liver function tests, blood urea nitrogen, bilirubin, complete blood count (CBC); concomitant or recent receipt of other investigational agents; expectation that we would not be able to monitor the patient for the duration of the study; mother with serology positive for hepatitis B virus surface antigen or neonate's receipt of hepatitis B virus immune globulin since birth.

Evaluation of patients. After informed consent was obtained from the infant's parents or legal guardian, a baseline evaluation of medical history, physical examination, and laboratory testing was performed. Laboratory evaluations included standard hematology, blood chemistry, liver function, renal function, and urinalysis testing.

Fifteen minutes before administration of the study drug, vital signs, oxygen saturation, and physical examination were obtained. The randomized dose of study drug (PAGIBAXIMAB or placebo) was administered as an i.v. infusion at 0.01 ml per kilogram per minute. The infusion rate was slowly increased to 0.02, 0.05, 0.1, and 0.125 ml/kg/min every 15 min if there was no physical evidence of an adverse event (AE), including changes in oxygen saturation, heart rate, blood pressure, temperature, and respirations. Additional vital signs and clinical assessment data were collected every 15 min until the infusion was complete and 30 and 60 min post-infusion.

On day 14, patients were rescreened for eligibility for the second dose. Eligible patients had a similar predose evaluation. Administration of the second dose of study drug occurred at the patient's previous randomized dose based on the original treatment assignment, and infusion followed the same procedures described for the initial infusion. On days 3 and 7, the patients were assessed for safety, medical history, vital signs, and physical examination. On days 14, 28, and 42, the patients were assessed for safety, medical history, vital signs, physical examination, and the following were obtained: urinalysis, CBC with differential and platelet count, aspartate aminotransferase, alanine aminotransferase, blood urea nitrogen, and creatinine Data on bilirubin, electrolytes, and glucose were also collected. Additionally, on day 42, blood was drawn for human anti-murine antibody/human anti-chimeric antibody (HAMA/HACA) analysis. On day 56, if still hospitalized, patients were assessed for safety, medical history, vital signs, physical examination, and urinalysis was obtained. If a patient was released from the hospital prior to day 56, the patient's parent or legal guardian was contacted by telephone and asked about the patient's health since hospital discharge. Blood was drawn for anti-LTA antibody levels on days 0 (1 h after infusion), 3, 7, 14 (prior to the second infusion), 14 (1 h after the second infusion), 28, 42, and 56 (if still hospitalized). Patients were monitored for a total of 8 weeks after the start of the first study drug infusion.

Throughout the study, patients were closely monitored for signs of infection. If the attending physician deemed it necessary to evaluate a patient for sepsis, meningitis, or other infection, a workup was performed. The workup included CBC with differential; platelet count; blood culture from a peripheral vein (although physicians were encouraged to collect two samples, a single sample was considered acceptable); cerebrospinal fluid examination, including gram stain, cell count, and culture; urine culture by bladder tap or sterile catheterization; and culture from other sterile sources as indicated. Samples of all staphylococcal bacteria isolated from blood, cerebrospinal fluid, and other sterile sites were sent to a central laboratory for analyses.

Randomization and dose escalation. Eligible VLBW infants were randomized at a ratio of 2:1 to receive i.v. PAGIBAXIMAB at 10, 30, 60, and 90 mg/kg or an equal volume of placebo (saline) on days 0 and 14. Two birth weight groups (700 to 1,000 g and 1,001 to 1,300 g) accrued independently, with each dose and birth weight group consisting of patients receiving PAGIBAXIMAB and patients receiving placebo. Dose escalation to the next higher level occurred within each birth weight group only after the last patient in the previous dose and weight group was monitored for at least 14 days from the first dose by the safety monitoring committee.

Blinding. This was a double-blind study. The only persons who knew patient treatment assignments were the statisticians at the contract research organization overseeing the study, who were responsible for assignment of patient identification numbers and study drug allocation, and the pharmacists at the study sites, who were responsible for preparing the infusion.

Safety analyses. For the purpose of this study, an AE was defined as any adverse change from the patient's baseline condition that occurred following the first administration of the study drug through the end of the study period. Changes from the patient's baseline condition known to be normal physiologic changes associated with the development of premature infants were not considered to be AEs. A protocol-defined toxicity table was used to grade the severity of each AE on a scale of 1 to 4.

A serious adverse event (SAE) was defined as any event that resulted in any of the following outcomes: (i) death; (ii) a life-threatening AE; (iii) inpatient hospitalization or prolongation of hospitalization; (iv) persistent or significant disability or incapacity; (v) an important medical event that required intervention in order to prevent any of the other serious outcomes. All grade 4 AEs, as defined in the toxicity table, were considered SAEs. The investigator assessed each SAE for severity and relationship to study drug in a blinded manner. Assessment of immunogenicity. HAMA/HACA levels were determined by radiometric human anti-Hu96-110 HACA assays. Hu96-110, a human/murine chimeric monoclonal antibody, is the active ingredient of PAGIBAXIMAB. Polystyrene beads were coated with Hu96-110. Test serum, normal human serum, and goat anti-human IgG (positive control) were added to borosilicate glass culture tubes. A single Hu96-110-coated bead was added to each tube. Each bead was washed. 125I-labeled Hu96-110 was added to all tubes. Each bead was washed again. The beads were transferred to clean tubes, and particle emissions were counted to determine the amount of 125I-labeled Hu96-110 bound to each bead. The assay result was calculated from the bound 125I-labeled Hu96-110 and known concentration of 125I-labeled Hu96-110. The results were expressed as nanograms per milliliter of Hu96-110. During validation of the assay, the response in normal human serum samples was 25.4±8.7 ng/ml. The upper limit of normal human serum was defined as the mean plus 3 standard deviations or 52 ng/ml. A positive sample would have a value that exceeds the upper limit and or was twice as high as the pre-infusion sample.

Pharmacokinetic analyses. The pharmacokinetic profile of PAGIBAXIMAB was assessed using an antigen capture enzyme-linked immunosorbent assay measuring the concentration of anti-LTA antibodies in serum. S. aureus LTA was coated onto the bottom of 96-well microtiter plates. After unbound LTA was washed off, test sera diluted in phosphate-buffered saline with Tween 20 and immunoglobulin-depleted human serum was incubated in the wells. The bound anti-LTA antibody was detected by incubation with a horseradish peroxidaselabeled anti-human immunoglobulin antibody and a colorimetric reagent (3,3′,5,5′-tetramethylbenzidine [TMB]). The amount of anti-LTA antibody in serum was determined by comparison to a PAGIBAXIMAB standard of known amount (Weisman et al. 2009. Int. Immunopharmacol. 9:639-644., herein incorporated by reference in its entirety). Noncompartmental analysis was used to estimate clearance, volume of distribution (V), and half-life (t_{1/2}).

Opsonophagocytic activity. The opsonophagocytic (bacterial killing) activity of PAGIBAXIMAB in serum was determined using a modified standard assay (Fischer et al. 1994. J. Infect. Dis. 169:324-329., herein incorporated by reference in its entirety). Specifically, the following components were used: Staphylococcus epidermidis American Type Culture Collection (ATCC) strain 55133 (for measurement of patient serum activity) or clinical isolates (for measurement of PAGIBAXIMAB activity) as the source of bacteria, HL60 cells (human acute promyelocytic leukemia cell line) as a source of human polymorphonuclear cells (PMNs), and C1q as a source of complement. Patient serum (diluted 1:90) or PAGIBAXIMAB (at various concentrations), PMNs, and diluted complement were mixed with a suspension of bacteria and incubated in a 96-well plate. Bacterial killing was measured by comparing the number of bacteria present at the time of initial mixing and after 2 h of incubation. Bacteria were enumerated by performing colony counts on tryptic agar plates with 5% sheep blood. Controls included PMNs alone, complement alone, and PMNs plus complement. Using the formula {[number of bacteria (time zero to 2 h)]/number of bacteria at time zero]} 100, the percent bacterial killing was calculated.

Bacterial analysis. Frozen stocks of staphylococcal isolates were shipped to a central laboratory for species identification. Clinical isolates of CONS from blood cultures were analyzed. The isolates were thawed and streaked for isolation on blood agar (Remel, Lenexa, Kans.) to confirm culture purity and presence of staphylococci. The species was determined by using the API Staph Ident system (BioMerieux, Hazelwood, Mo.) (Kloos & Wolfshohl. 1982. J. Clin. Microbiol. 16:509-516., herein incorporated by reference in its entirety). Isolated staphylococcal colonies were tested in various biochemical assays per the manufacturer's instructions. Two ATCC reference isolates, ATCC 49521 and ATCC 35984, were used as control organisms for S. aureus and S. epidermidis, respectively. Those staphylococcal isolates for which an unequivocal species could not be determined by API Staph Strip, were sent to Accugenix (Newark, Del.) for 16S 500-bp sequence identification. The same isolates were evaluated for genetic relatedness by performing pulsed-field gel electrophoresis (PFGE) (Raimundo et al. 2002. Hosp. Infect. 51:33-42., herein incorporated by reference in its entirety). Chromosomal DNA was isolated from the various staphylococcal isolates, digested in agarose with SmaI, and then subjected to PFGE using a contour-clamped homogeneous electric field system (Bio-Rad, Hercules, Calif.). Dendrograms were generated based on the genetic relatedness of the digestion patterns (McDougal et al. 2003. J. Clin. Microbiol. 41:5113-5120., herein incorporated by reference in its entirety).

Analysis of sepsis episodes. For all patients who had sepsis evaluations, analyses of sepsis caused by CONS were divided into four categories. Each category included signs and symptoms consistent with clinical sepsis. In addition, if two or more peripheral blood cultures grew CONS, it was categorized as definite sepsis. If one peripheral blood culture grew CONS when only one peripheral blood culture was drawn, it was categorized as probable sepsis. If one peripheral blood culture grew CONS when more than one peripheral blood culture was drawn, it was categorized as possible sepsis. If one or more central venous line blood cultures grew CONS in the absence of positive peripheral cultures, it was categorized as line sepsis.

Statistical methods. The statistical analyses were essentially descriptive. Safety analyses were performed on the intent-to-treat (ITT) population, defined as all randomized patients who received at least one dose of study drug. Continuous variables were summarized by the mean, standard deviation, median, and range. Categorical variables were summarized by the frequency and percentage.

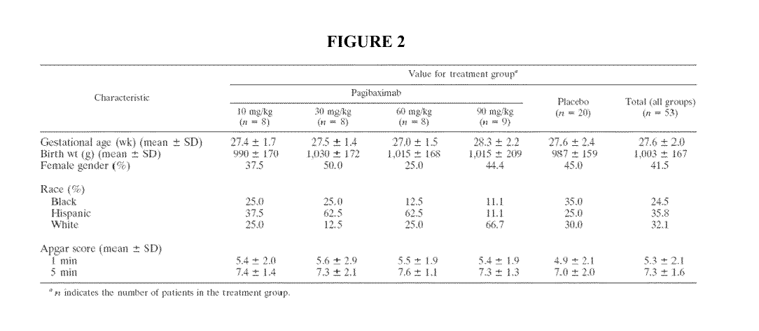

Patient baseline characteristics. Demographic and other baseline characteristics of study patients were generally comparable across the treatment groups (SEE FIG. 2). The mean gestational age for patients was 27.6 weeks (ranging from 25.0 to 33.0 weeks), and the mean birth weight was 1,003 g (ranging from 702 to 1,300 g).

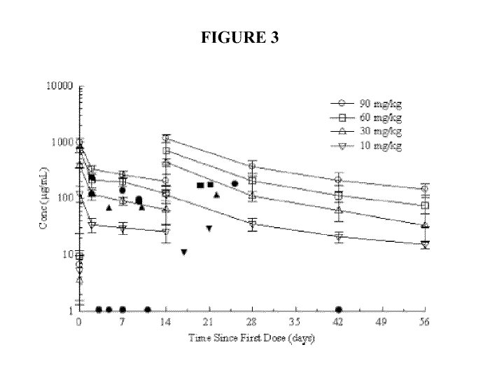

Pharmacokinetics. Mean patient preinfusion (endogenous) plasma anti-LTA concentrations were low and ranged from 3.49 to 9.44 μg/ml across the dose groups. Mean plasma anti-LTA concentrations increased in a dose-related manner (SEE FIG. 3). In the 60-mg/kg and 90-mg/kg dose groups, following the second infusion of PAGIBAXIMAB, an extrapolation of the serum antibody levels suggests that a sustained mean anti-LTA level over 500 μg/ml was observed for a period of approximately 6 and 12 days, respectively (SEE FIG. 2). 85.7% of the patients in the 60-mg/kg dose group and 100% of the patients in the 90-mg/kg dose group had plasma anti-LTA concentrations over 500 μg/ml immediately after the day 14 PAGIBAXIMAB infusion. In the 10-mg/kg and 30-mg/kg dose groups, no patient and 0% and 12.5% of the patients, respectively, had an antibody concentration over 500 μg/ml immediately after the day 14 infusion. One patient in the 90-mg/kg dose group had an antibody concentration over 500 μg/ml on day 28. Following i.v. infusion of PAGIBAXIMAB, mean pharmacokinetic values across dose groups were independent of dose. Total plasma CL ranged from 0.32 to 0.43 ml per hour, mean V ranged from 182 to 285 ml, and mean t_{1/2} ranged from 369 to 599 h or 19 to 25 days. The pharmacokinetics of PAGIBAXIMAB in premature infants therefore appeared linear at doses ranging from 10 to 90 mg/kg.

Examination of the mean plasma anti-LTA over time profile (SEE FIG. 3) indicated that the decay after the first dose appeared to be similar to that after the second dose.

Dose proportionality analysis showed that the linear regression of the log area under the plasma drug concentration-time curve versus the log total dose suggested that for these data, the doses were proportional with the estimated slope of 0.92, and the 95% confidence interval of 0.78 to 1.06.

A patient in the 90-mg/kg dose group received only the first dose of PAGIBAXIMAB, but blood samples were collected for the entire 56-day period. This patient's pharmacokinetic parameters for CL (0.347 ml/h), V (241 ml), and t_{1/2} (481 h) were consistent with the rest of the 90-mg/kg group, suggesting that the pharmacokinetics of PAGIBAXIMAB were consistent after one or two doses.

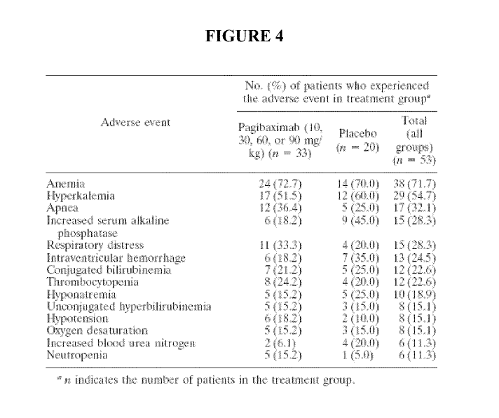

Adverse events. 98% of the patients in the ITT population experienced at least one AE during the study. AEs experienced by patients in this study were consistent with events known to occur with prematurity and in low-birth weight neonates (Avery et al. 1999. Neonatology: pathophysiology and management of the newborn, 5th ed., p. 1501-1534. Lippincott Williams & Wilkins, Philadelphia, Pa., herein incorporated by reference in its entirety). The AEs most commonly reported in study patients were anemia and hyperkalemia, with 71.7% and 54.7% of the patients, respectively, experiencing at least one episode. The percentages of patients experiencing the most common AEs were generally similar in the treatment groups (SEE FIG. 4). There was no trend toward increased frequency of clinical or laboratory AE with increased PAGIBAXIMAB dose.

One AE, moderate oxygen supplementation, was assessed by the investigator as probably related to study drug infusion. This event, experienced by a patient in the 90-mg/kg PAGIBAXIMAB group, occurred immediately after the second study drug infusion and resolved in 1 h. All other AEs were considered by the investigators as either unrelated or probably not related to study drug.

Serious adverse events. 43% of the patients in the ITT population experienced at least one SAE during the study. Cholestasis was the most common SAE, with 18.9% of patients reporting at least one episode. Other SAEs occurring in ≧5% of patients in the ITT population included necrotizing enterocolitis (NEC) and sepsis due to an identified organism (13.2% each), and hyperkalemia and thrombocytopenia (5.7%, each).

The SAEs experienced by patients in this study were generally similar across treatment groups. No trend toward increased frequency of SAEs with increasing PAGIBAXIMAB dose was observed. All SAEs reported for patients in this study were recognized comorbidities associated with prematurity, and all were assessed by the investigators as either unrelated or probably not related to study drug.

Significant clinical outcomes. In order to assess any potential adverse effect of PAGIBAXIMAB on clinical events known to occur at high frequency in low-birth-weight neonates, the frequency of patients experiencing NEC (Bell's stage 2 or greater) (Bell et al. 1978.

Ann. Surg. 187:1-7., herein incorporated by reference in its entirety), bronchopulmonary dysplasia (oxygen dependency at 36 weeks postmenstrual age) (Bancalari et al. 2003. Semin. Neonatol. 8:63-71., herein incorporated by reference in its entirety), severe intraventricular hemorrhage (Papille's grade 3 or 4) (Papile et al. 1978. J. Pediatr. 92:529-534., herein incorporated by reference in its entirety), retinopathy of prematurity requiring surgery, and death were summarized by treatment group. The percentages of patients in the PAGIBAXIMAB and placebo treatment groups experiencing significant clinical outcomes were generally similar for bronchopulmonary dysplasia (57.6% versus 66.6%, respectively), NEC (15.2% versus 11.1%, respectively), retinopathy of prematurity requiring surgery (3.0% versus 10.0%, respectively), and death (9.1% versus 5.0%, respectively). A severe intraventricular hemorrhage was experienced by 3.0% of patients in the PAGIBAXIMAB group and 20% of patients in the placebo group. The number of patients experiencing significant clinical outcomes in the individual treatment groups was small; however, no trend toward increased frequency of any significant clinical event with increased PAGIBAXIMAB dose was observed.

Deaths. 7.5% of the patients in the ITT population died during the study, including 9.1% of the patients receiving PAGIBAXIMAB and 5.0% of the patients receiving placebo. A second patient in the placebo group died 7 months after completing the study follow-up period.

One patient in the PAGIBAXIMAB treatment group died on study day 21 due to NEC and sepsis. This infant received the first dose of PAGIBAXIMAB (10 mg/kg) on study day 0 and did not receive the second dose because of failure to fulfill the eligibility criteria. A second patient in the PAGIBAXIMAB treatment group died on study day 5 due to severe hyaline membrane disease and subsequent NEC, and no organism was identified. This infant received the first dose of PAGIBAXIMAB (10 mg/kg) on study day 0 and died prior to receiving the second dose. A third patient in the PAGIBAXIMAB treatment group died on study day 11 from sepsis. This infant received the first dose of PAGIBAXIMAB (60 mg/kg) on study day 0 and died prior to administration of the second dose.

One patient in the placebo group died on study day 36 from sepsis, NEC, and prematurity resulting in multiple organ failure. This infant received the first dose of placebo (as part of the 10-mg/kg dose group) on study day 0 and did not receive the second dose because of failure to fulfill the eligibility criteria. A second patient in the placebo group died 7 months after completing the study follow-up period. The immediate cause of death was cardiopulmonary failure secondary to multiple organ system insufficiency and extreme prematurity. None of these deaths was considered by the investigators to be attributable to study drug. All of the events resulting in death are known to be associated with premature infants with very low birth weight.

HAMA/HACA analysis. Concentrations of HAMA/HACA were relatively unchanged for all patients across treatment groups throughout the study and remained well below the upper normal limit (52 ng/ml) from predose to postdose.

Vital signs, physical examinations, and clinical chemistry/hematology/urinalysis. In all treatment groups, patient infusion vital signs showed normal variability. Noninfusion vital signs showed no indication of a dose response effect. Systolic pressure and diastolic pressure increased with age, as expected for this population, and were similar across treatment groups. Heart rate and respiratory rate showed normal variability for all treatment groups. Temperature was stable over time for all treatment groups. The median body weight increased from approximately 1,000 to 2,140 g over the study period; all dose groups showed the same tendency. Variability in all laboratory results over time was consistent with premature newborn parameters.

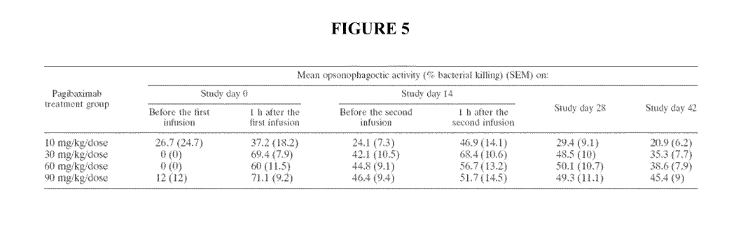

Opsonophagocytic activity. Pagibaximab enhanced the opsonophagocytic (bacterial killing) activity in serum (SEE FIG. 5). An increase in opsonophagocytic activity was demonstrated at the lowest dose level (10 mg/kg) and was increased at the higher dose levels. There did not appear to be a significant difference in activity between the 30-, 60-, and 90-mg/kg groups. Minimal or no opsonophagocytic activity was observed in patients treated with placebo.

Clinical signs and symptoms leading to evaluation of sepsis. Sepsis evaluations were performed in 96% of the patients in the ITT population. The most common signs and symptoms leading to evaluation for sepsis were similar across treatment groups. The most common clinical signs and symptoms leading to evaluation of sepsis were similar for patients in the PAGIBAXIMAB and placebo treatment groups, with apnea/bradycardia accounting for 23.7% and 26.2% of events, respectively, and cyanosis accounting for 18.3% and 18.5% of events, respectively. Overall, no dose response effect upon the frequency of signs and symptoms leading to evaluation of sepsis was observed.

Sepsis. 50.9% of patients, including 48.5% patients in the PAGIBAXIMAB treatment group and 55% patients in the placebo treatment group, experienced at least one sepsis episode. 9.1% of patients in the PAGIBAXIMAB treatment group and 15% of the placebo treatment group experienced a second episode of sepsis. Patients each in the PAGIBAXIMAB treatment group (12.1%) and the placebo treatment group (20%) experienced sepsis with multiple organisms. Coagulase-negative staphylococcus was the most common organism (40.5%) isolated from blood cultures in patients with sepsis in both the PAGIBAXIMAB and placebo treatment groups, and only one patient in the 60-mg/kg PAGIBAXIMAB group experienced Staphylococcus aureus (2.4%) sepsis as part of a mixed infection with CONS (SEE FIG. 3). Nonstaphylococcal sepsis events occurred in both the PAGIBAXIMAB (21.2%) and placebo (45%) treatment groups. The organisms isolated from these blood cultures were Enterococcus (14.3%), Candida (7.1%), Escherichia coli (7.1%), Klebsiella (7.1%), Pseudomonas (7.1%), Enterobacter (4.8%), Serratia (4.8%), Acinetobacter (2.4%), and Streptococcus agalactiae (2.4%) and did not differ significantly between groups.

Sepsis caused by CONS. 31% of patients experienced sepsis caused by CONS, including 33.3% of the patients receiving PAGIBAXIMAB and 25% of the patients receiving placebo. 9.1% of the patients in the 30-mg/kg PAGIBAXIMAB group experienced a second episode of sepsis caused by CONS. Although analysis by PAGIBAXIMAB dose level showed a slightly greater proportion of patients in the 90-mg/kg PAGIBAXIMAB group experiencing sepsis caused by CONS (44%) compared with those in the other treatment groups, statistical testing using Fisher's exact test showed no overall difference between dose groups. Of the patients with sepsis caused by CONS, 63% experienced definite sepsis and 31.3% probable sepsis. No patient experienced possible sepsis caused by CONS. One patient receiving PAGIBAXIMAB at the 90-mg/kg dose level experienced line sepsis caused by CONS. In all cases, estimated or observed plasma anti-LTA levels were below the putative protective level of 500 μg/ml at the time of diagnosis of CONS-caused sepsis. The species identification of the isolates in the patients with sepsis caused by CONS revealed substantial variation with sepsis caused by S. epidermidis in 69% of patients, and in 6.2% of patients each by Staphylococcus simulans, Staphylococcus caprae, mixed infection of S. epidermidis and Staphylococcus hominus, mixed infection of S. epidermidis and Staphylococcus haemolyticus, and mixed infection of S. epidermidis and S. aureus.

Age at diagnosis of first episode of sepsis caused by CONS. The mean age of patients at the diagnosis of the first episode of sepsis caused by CONS ranged from 11.5 to 22.5 days across treatment groups. In the 10-mg/kg PAGIBAXIMAB treatment group, the mean age at diagnosis of the first episode was 22.5 days, in the 30-mg/kg treatment group, it was 11.5 days, in the 60-mg/kg treatment group, it was 16.0 days, and in the 90-mg/kg treatment group, it was 16.0 days. In the placebo treatment group, the mean age at first diagnosis of CONS sepsis was 17.8 days.

Opsonizability of CONS by PAGIBAXIMAB. Of 25 staphylococcal isolates recovered from the blood cultures of 16 patients with staphylococcal infection, PAGIBAXIMAB demonstrated bacterial killing (opsonophagocytic assay) against all the isolates. However, there was distinct heterogeneity in the ability of antibody to opsonize the different isolates. Whereas some isolates were opsonized at a concentration of less than 50 μg/ml, others required 400 μg/ml. At PAGIBAXIMAB concentrations of 400 μg/ml, 67% of isolates demonstrated ≧90% bacterial killing, 78% of isolates demonstrated ≧80% bacterial killing, and 89% of isolates demonstrated ≧70% bacterial killing.

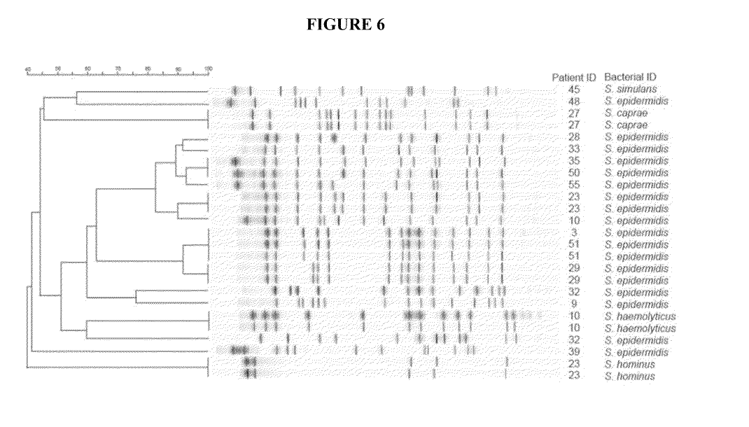

Dendrogram of CONS. CONS bacterial isolates were analyzed for genetic relatedness. The dendrogram (SEE FIG. 6) of these isolates (using a similarity coefficient of 80%) suggests that the strains of CONS varied substantially and were generally unrelated between patients who were infected, even in the same hospital (data not shown). There appeared to be two clusters that were closely related: patients 28, 33, 35, 50, 55, 23, and 10 from the same hospital and patients 3, 51, and 29 from two different hospitals in New York, N.Y., and Houston, Tex. When paired cultures for the same sepsis episode were tested, all of these pairs appeared to be related. Although one patient (patient 23) appeared to have two species in each culture, the strains in the two cultures appeared related. The other patient (patient 10) appeared to have two species in one culture and one species in the other culture, and the species in the two cultures appeared to be related.

Discussion. Mean preinfusion (endogenous) plasma anti-LTA antibody levels were found to be negligible in VLBW neonates. This may be because premature infants do not receive their normal transplacental passage of antibody which occurs predominantly in the final weeks of pregnancy (Ballow et al. 1986. Pediatr. Res. 20:899-904., herein incorporated by reference in its entirety), or the immaturity of the premature neonatal immune system makes it unlikely that they would mount a significant antibody response following exposure in utero or in the first few days of life (Strunk et al. 2007. Neonatal immune responses to coagulase-negative staphylococci. Curr. Opin. Infect. Dis. 20:370-375., herein incorporated by reference in its entirety), although the present invention is not limited to any particular mechanism of action and an understanding of the mechanism of action is not necessary to practice the present invention. A direct correlation between low serum levels of IgG and an increased risk of late-onset neonatal sepsis has been shown (Fanaroff et al. 1998. Pediatr. Infect. Dis. J. 17:593-598., Gladstone et al. 1990. Pediatr. Infect. Dis. J. 9:819-825., herein incorporated by reference in their entireties). Thus, VLBW neonates are unlikely to possess functional opsonophagocytic activity to staphylococci at birth or during hospitalization, making them a population at high risk of staphylococcal sepsis. Therefore, passive immunization with PAGIBAXIMAB is an important step in preventing neonatal staphylococcal infection. This is especially important in methicillin (meticillin)-resistant (community and hospital acquired) and methicillin-sensitive S. aureus infections, which although these infections occur less frequently than CONS infections, they result in greater morbidity and mortality than CONS infection in the premature infant (DeJonge et al. 2007. J. Pediatr. 151:260-265., Derkx et al. 1999. Clin. Infect. Dis. 28:770-777., Stoll et al. 2002. Pediatrics 110:285-291., herein incorporated by reference in their entireties).

The pharmacokinetics of PAGIBAXIMAB in premature neonates was dose proportional over doses ranging from 10 to 90 mg/kg. The mean t_{1/2} ranged from 19 to 25 days across the dose groups and is consistent with previous reports of IgG infusions in neonates (35, 36). This is also consistent with results from a previous study of PAGIBAXIMAB in healthy human adults (38), with other studies of human/mouse chimeric or full human IgG1 antibodies administered i.v. in adults (Azuma et al. 1991. J. Immunother. 10:278-285., Chow et al. 2002. Clin. Pharmacol. Ther. 71:235-245., LoBuglio et al. 1989. Proc. Natl. Acad. Sci. USA 86:4220-4224., Solomon et al. 1963. J. Lab. Clin. Med. 62:1-17., herein incorporated by reference in their entireties), and commercially available intravenous immunoglobulin in neonates (Weisman et al. 1986. Pediatr. Infect. Dis. J. 5(3 Suppl.):S185-S188., Weisman et al. 1989. Vox Sang. 57:243-248., herein incorporated by reference in their entireties). Moreover, after the second infusion of PAGIBAXIMAB at 60 or 90 mg/kg, mean anti-LTA levels greater than 500 μg/ml, the putative protection level for staphylococcal sepsis, were observed. With the 90-mg/kg dose achieving more sustained levels greater than 500 μg/ml.

HAMA/HACA levels remained low in the neonates receiving i.v. PAGIBAXIMAB at 10, 30, 60, and 90 mg/kg at study days 0 and 42, indicating that PAGIBAXIMAB does not elicit an antibody response to itself even after repeated doses. In addition, the AEs, SAES, and clinical outcomes across study groups were not significantly different. This is also similar to previous reports of IgG safety and tolerability in neonates. Experiments conducted during development of embodiments, of the present invention indicate that the first use of PAGIBAXIMAB in VLBW neonates at 10, 30, 60, and 90 mg/kg administered i.v. twice 2 weeks apart appeared safe and well tolerated.

CONS was the most common cause of sepsis in the VLBW neonates in this study, with an incidence of 30.2% across treatment groups, and less than 2% of patients developed S. aureus sepsis. These findings are consistent with the results of previous larger studies of late-onset sepsis in VLBW infants (Aziz et al. 2005. BMC Pediatr. 5:22., DeJonge et al. 2007. J. Pediatr. 151:260-265., Stoll et al. 2002. Pediatrics 110:285-291, herein incorporated by reference in their entireties) that demonstrated CONS in 14 to 23% of patients and S. aureus in 1.6 to 5% of patients. The majority (63%) of sepsis cases caused by CONS were confirmed by two or more peripheral blood cultures growing CONS. There was no significant difference in incidence rates of sepsis caused by CONS across dose levels of PAGIBAXIMAB and placebo, overall or by category of infection.

Patients receiving 60 or 90 mg/kg of PAGIBAXIMAB were observed to have sustained plasma anti-LTA levels above the putative protection level of 500 μg/ml following the second dose. At the time of diagnosis of sepsis caused by CONS, all affected patients had estimated or observed plasma anti-LTA levels below 500 μg/ml.

### Example 3

### Population Modeling to Guide Dosing of Anti-LTA Antibody Composition in Very Low Birth Weight (VLBW) Premature Infants

Anti-LTA antibody composition (PAGIBAXIMAB, PAG), an anti-staphylococcal monoclonal antibody evaluated for prevention of staphylococcal sepsis in VLBW, (weight <1200 gm) in a phase 2 study reported no staphylococcal sepsis at serum levels ≧500 ug/ml (See Examples 1 and 2 above). Thus, experiments were conducted during development of embodiments of the invention in order to identify and characterize a dose scheme that would maintain levels of anti-LTA antibody composition ≧500 μg/ml in a phase 3 efficacy study.

Methods: pharmacokinetic (PK) data from 100 VLBW enrolled in multicenter placebo controlled 1 Phase 1 and 2 studies who were infused with 10 to 90 mg/kg Anti-LTA antibody composition (PAGIBAXIMAB) for one to three doses were used to develop a PK model. Simulation was used to select a dosing regimen. The regimen was prospectively evaluated in an ongoing double blind multicenter placebo controlled Phase 2/3 study using PK samples collected over 35 days.

Experiments conducted during development of embodiments of the invention found that the best model to describe the concentration time course of anti-LTA antibody compositions was a two compartment open model with first order elimination from the central compartment. The model included a sub-model for the time course of endogenous substrate that was described using a baseline value with an exponential fall-off because the endogenous substance appeared to decrease over time.

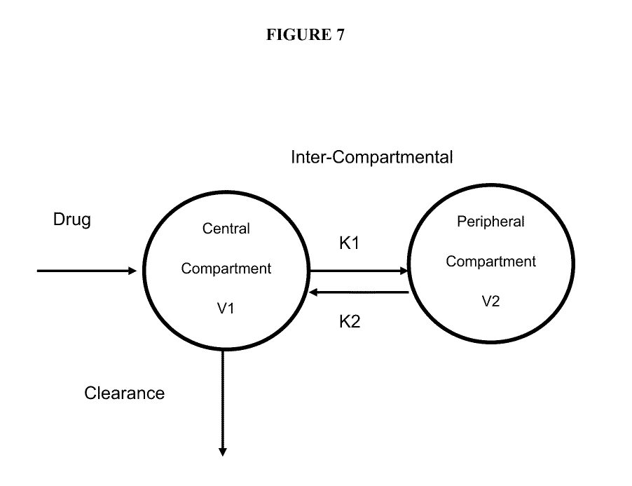

The model was parameterized for clearance (CL), the volume of distribution of the central compartment (V1), inter-compartmental clearance (Q), and the volume of distribution of the peripheral compartment (V2). In addition, the submodel developed for the endogenous substance (C0) was parameterized for a baseline value (C0int) and a rate constant of loss (Ke). 23 patients randomized to placebo had measurable concentrations over the sampling interval, and the Day 0 predose concentration was measurable for many patients randomized to active treatment. Terms for inter-individual variance were included for CL, V1, V2 and C0int and Ke. A full covariance matrix for CL, V1 and V2 was included. A schematic of this model is given in FIG. 7. The parameters that were estimated for the model are provided in Table 1, below:

TABLE 1
Parameter Estimates from Final Assessment of MAB-N002 and MAB-
N003

| | | Between | Between |
|---|---|---|---|
| | Typical Parameter | Patient | Occasion |
| Parameter (Units) | Value (se) | Variability | Variability |
| CL (mL/h) | 0.446 (4.3) | 24.3 | 8.81 |
| Effect of Weight | 0.75 FIX | | |
| Effect of Gest. Age | −1.53 (25.6) | | |
| V1 (mL) | 75 (4.82) | 22.4 | NE |
| Effect of Weight | 1.0 FIX | | |
| Q (mL/h) | 2.55 (16.3) | NE | NE |
| Effect of Weight | 0.75 FIX | | |
| V2 (mL) | 138 (3.8) | 12.4 | NE |
| Effect of Weight | 1.0 FIX | | |
| C0int (ug/mL) | 12.3 (16.6) | 42.8 | 16.0 |
| Effect of Study | 0.373 (68.8) | | |
| Ke | 0.000836 (66.5) | 66.5 FIX | NE |

| | |
|---|---|
| CCV Residual Error | 18.8 |
| (% CV) | |
| Additive Error (ug/mL) | 4.82 |

NE—not evaluated

Data were initially available from 24 patients, of which 13 patients had no measureable concentrations. Therefore data from a total of 11 patients were evaluated in this initial assessment. There were a total of 47 pharmacokinetic observation records available from these patients, 32 samples were planned pharmacokinetic draws and 15 were scavenged samples. A summary of the demographic data for these patients is provided in Table 2.

TABLE 2
Summary Demographics of Patients with Evaluable Data

| | AGE at | | WT at Birth | |
|---|---|---|---|---|
| | start (days) | AGE at end (days) | (kg) | Gender |
| N | 11 | 11 | 11 | Male = 8 |
| Mean | 3 | 14.2 | 0.936 | Female = 3 |
| SD | 0.6 | 8.9 | 0.163 | |
| Min | 2 | 4 | 0.65 | |
| Median | 3 | 11 | 0.975 | |
| Max | 4 | 26 | 1.187 | |

Two methods were used to evaluate the data and to determine if the pharmacokinetic behavior of anti-LTA antibody composition matched the expected behavior based on the prior evaluation. First, observed data were overlaid into the prediction intervals of concentration time data generated by simulation from the model developed previously. This provided a visual assessment of the performance of the model relative to the observed data.

The second approach was to fit the observed data using the previously developed model. In order to assess the utility of the scavenged samples, the model was fit with all available observations and with only planned observations and the results were compared. Because of the number of subjects (n=11) available for this interim evaluation, the First Order method was used. The parameters describing the time course of the endogenous concentration were fixed to the values that had been estimated from the previous analysis. The effect of study was removed from the model. Inter-occasion variability was also removed as it had been low in the initial assessment. Parameters from the estimation of the new data with 1) all observations and 2) only planned observations were compared to the previously estimated parameter values.

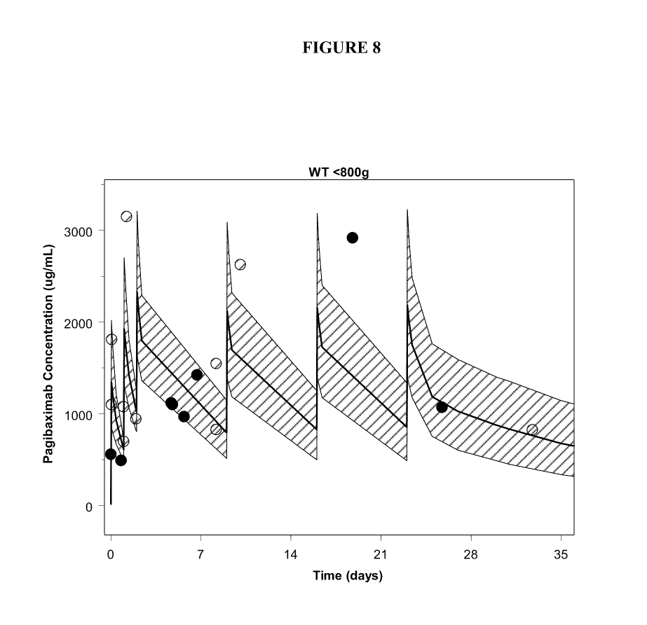

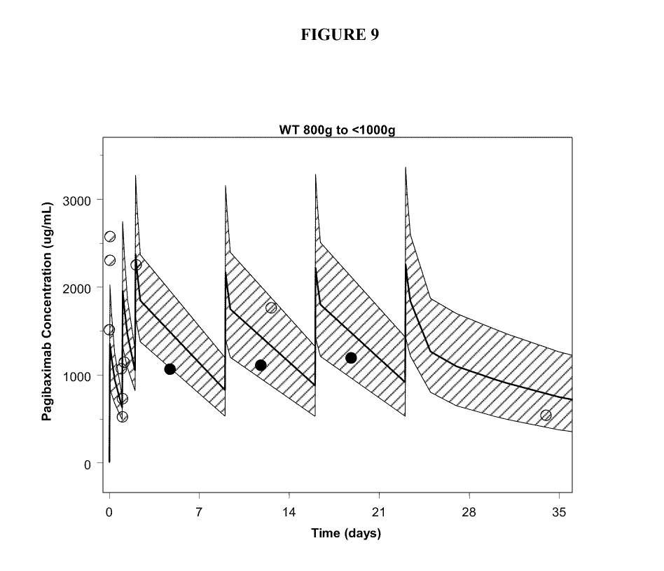

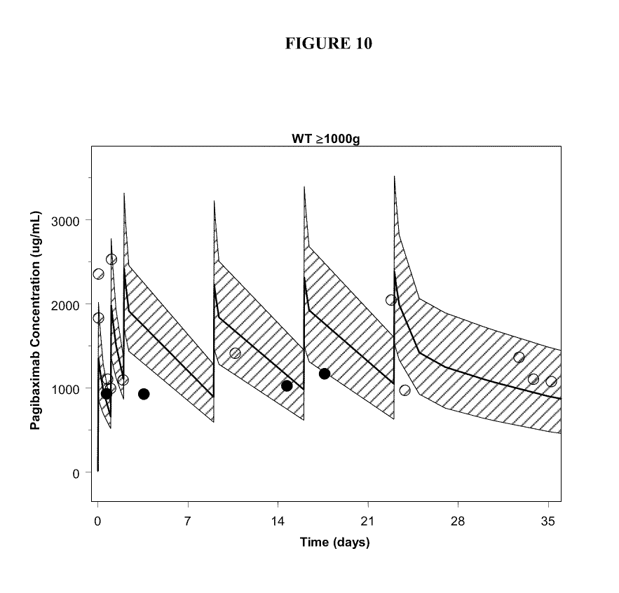

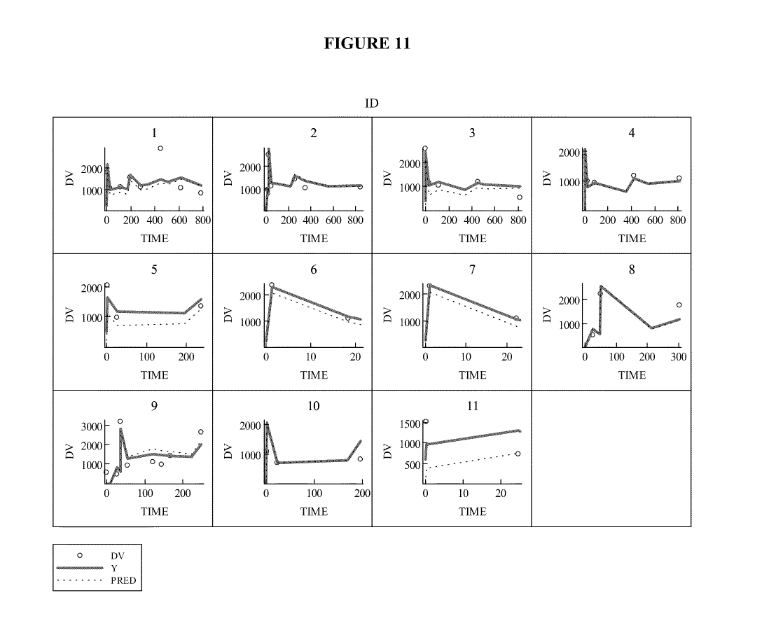

Observed Data Overlaid on Prediction Interval. Observed data from scavenged (filled circles) and planned (open circles) were overlaid on the simulated range of concentrations (i.e. 95% prediction intervals) by weight group. These prediction intervals were stratified by patient weight. Patients with weight less than 800 g (FIG. 8), weight from 800 to 1000 g (FIG. 9) and weight 1000 g or higher (FIG. 10) showed good agreement with the simulated intervals with most observed data being contained within the ranges of expected concentrations. Both planned and scavenged samples appeared to be within the expected range of concentrations, suggesting that both types of pharmacokinetic observation provided useful information on drug exposure. Overall the simulations generated from the developed model appeared to predict the concentrations in these new patients.

Model Estimation—All Observations. The results obtained from fitting all observations (planned and scavenged) to the previously developed model showed generally consistent results, although there were differences in the parameter estimates. The parameter estimates and associated 95% confidence intervals from the bootstrap are presented in Table 3. Clearance (CL) was 22% lower than the previous estimated value. The volume of distribution of the central compartment (V1) was 76% lower than the original estimate. The estimate of the between patient variability for clearance was also unexpectedly low. Confidence intervals for these parameters developed from estimating 1000 non parametric bootstrap replicates show very wide intervals for these parameters, suggesting that the values are not well defined. The mean estimated terminal half life from fitting both the planned and scavenged data was 14.5 days which was in good agreement from the previous estimate of half life of 15.4 days.

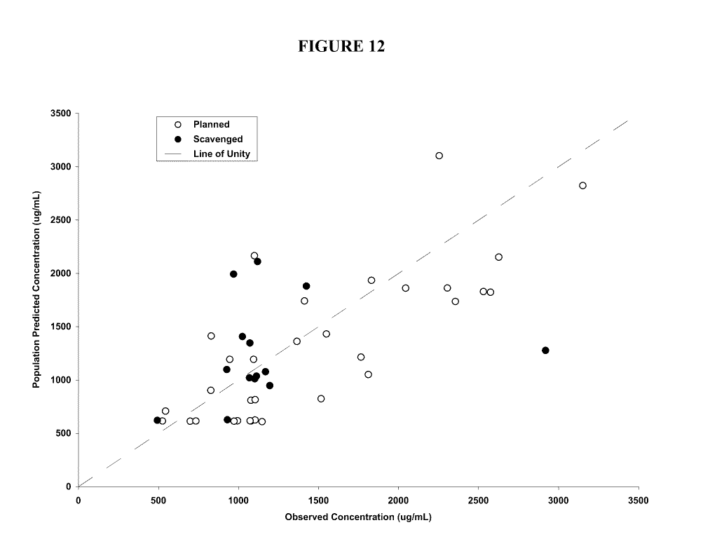

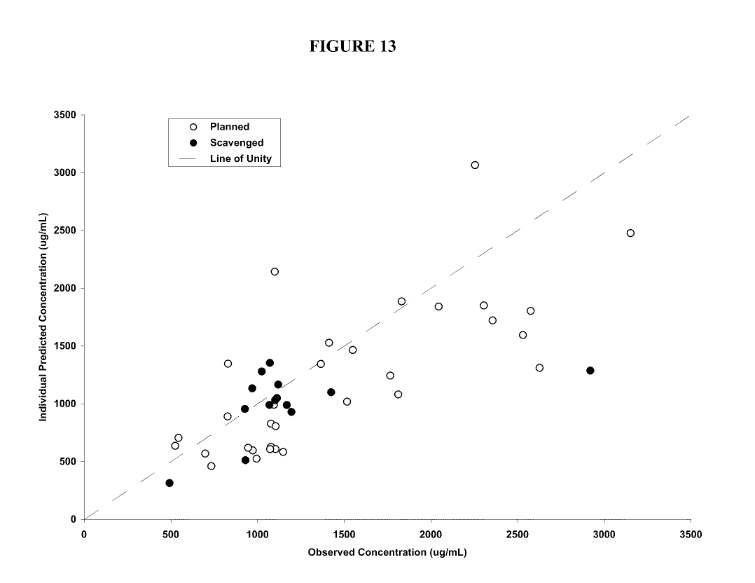

The individual (dummy blinded) plots of observed (open circles), typical predicted (black dashed lines) and individual predicted (solid lines) concentrations versus time show good agreement and the diagnostic plots of observed versus predicted (FIG. 12) and observed versus individual predicted (FIG. 13) concentrations show good model performance. There are no overt problems with the scavenged samples in these plots.

TABLE 3
Parameter Estimates - All Observations

| | | Between | Between |
|---|---|---|---|
| | Typical Parameter | Patient | Occasion |
| Parameter (Units) | Value (CI*) | Variability | Variability |
| CL (mL/h) | 0.35 (0.0324-0.474) | 2.05 (0-877) | NE |
| Effect of Weight | 0.75 FIX | | |
| Effect of Gest. Age | −1.53 FIX | | |
| V1 (mL) | 17.5 12.03-89.95) | 36.6 (5.98-225) | NE |
| Effect of Weight | 1.0 FIX | | |
| Q (mL/h) | 37.7 (1.52-49.65) | NE | NE |
| Effect of Weight | 0.75 FIX | | |
| V2 (mL) | 137 (71.86-347.6) | 44.9 (7.31-354) | NE |
| Effect of Weight | 1.0 FIX | | |
| C0int (ug/mL) | 12.3 FIX | 42.8 FIX | NE |
| Effect of Study | 0 FIX | | |
| Ke | 0.000836 FIX | 66.5 FIX | NE |

| | |
|---|---|
| CCV Residual Error | 34.6 |
| (% CV) | |
| Additive Error | 0.05 |
| (ug/mL) | |

However when the scavenged samples were fit with a different residual error the results were somewhat improved. The parameters and associated 95% bootstrap confidence intervals for this evaluation are presented in Table.

TABLE 4
Parameter Estimates N007 - All Observations Separate Residual Error
for Scavenged Samples

| | | Between | Between |
|---|---|---|---|
| | Typical Parameter | Patient | Occasion |
| Parameter (Units) | Value (CI*) | Variability | Variability |
| CL (mL/h) | 0.323 (0.0372-0.534) | 3.30 (2-162) | NE |
| Effect of Weight | 0.75 FIX | | |
| Effect of Gest. Age | −1.53 FIX | | |
| V1 (mL) | 15.4 (13.23-82.65) | 105 (1.8-203) | NE |
| Effect of Weight | 1.0 FIX | | |
| Q (mL/h) | 30.1 (1.64-47.58) | NE | NE |
| Effect of Weight | 0.75 FIX | | |
| V2 (mL) | 110 (67/32-313) | 11.2 (1.79-428) | NE |
| Effect of Weight | 1.0 FIX | | |
| C0int (ug/mL) | 12.3 FIX | 42.8 FIX | NE |
| Effect of Study | 0 FIX | | |
| Ke | 0.000836 FIX | 66.5 FIX | NE |

| | |
|---|---|
| CCV Residual Error | 34.6 |
| (% CV) | |
| Additive Error | 0.05 |
| (ug/mL) | |

Model Estimation—Planned Observations. When the subset of only planned observations (32 samples from 11 patients) was fit using the previous models, the results were surprisingly better than those obtained from the fit of all data (scavenged and planned). The estimate of clearance is similar to the estimate from the fit of all data and is 33% lower than the previous estimate. However the confidence intervals are narrower for this estimate than the estimate obtained for all data. The estimate of the central volume of distribution is 39% lower than the previous estimate but is in better agreement than the estimate obtained using all data. Estimated between patient variability is also consistent with previous estimates, albeit somewhat lower than before. The mean estimated terminal half life from fitting only the planned data was 16.7 days which was in good agreement from the previous estimate of half life of 15.4 days. The present estimated value for half life is also consistent with the value of half life that was estimated when the previous model was used to conduct a maximum a posteriori evaluation (half life=16.2 days) suggesting that the estimates from the planned data may be more reliable than the results using the scavenged data.

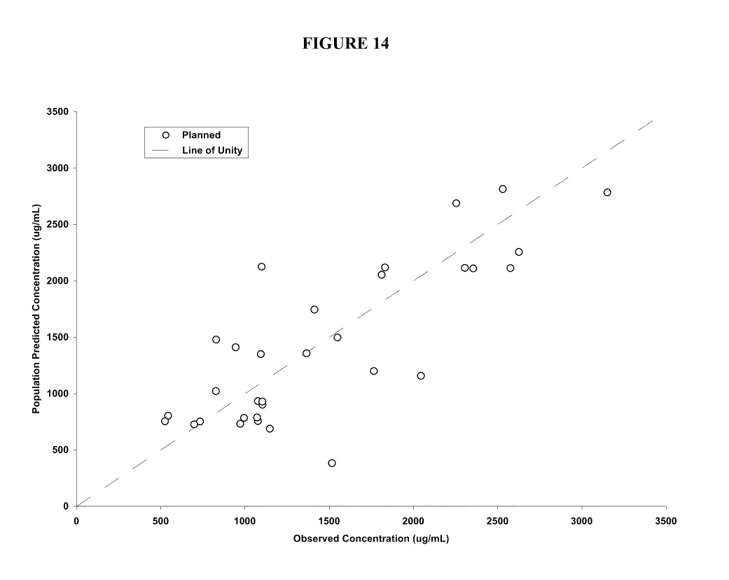

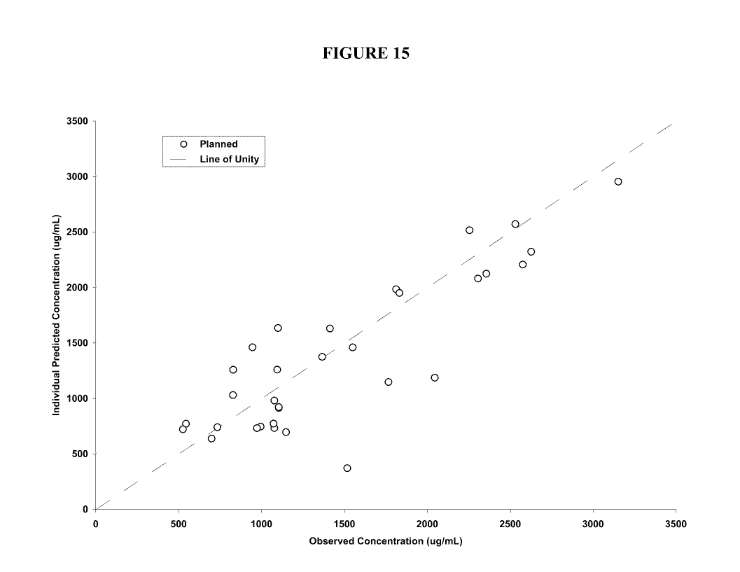

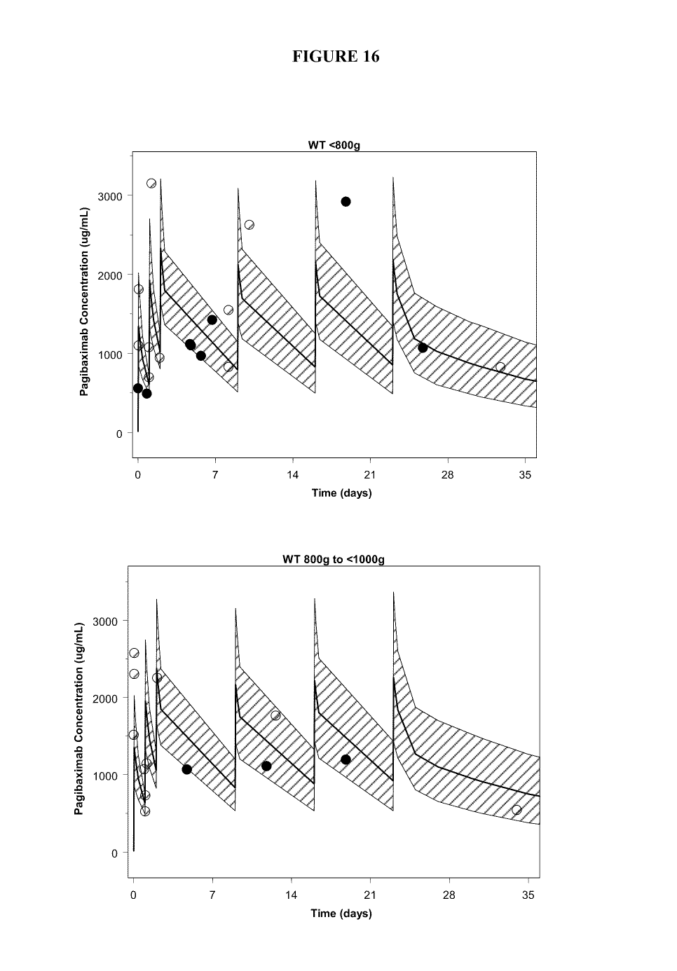

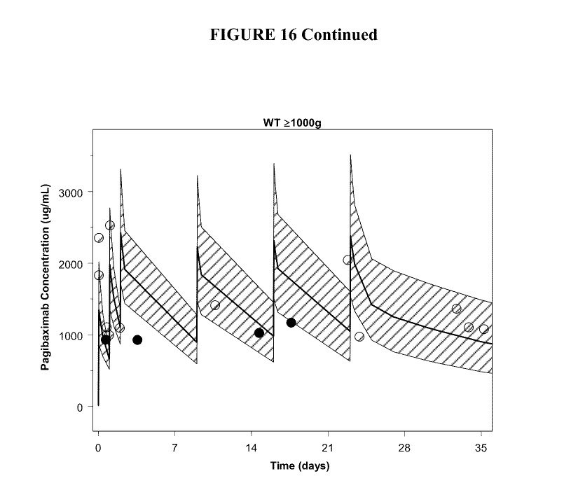

Diagnostic plots of observed versus predicted (FIG. 14) and observed versus individual predicted (FIG. 15) concentrations show good model performance.

TABLE 1
Parameter Estimates N007 - Planned Observations

| | | Between | Between |
|---|---|---|---|
| | Typical Parameter | Patient | Occasion |
| Parameter (Units) | Value (CI*) | Variability | Variability |
| CL (mL/h) | 0.298 (0.18-0.438) | 10.1 (0-101) | NE |
| Effect of Weight | 0.75 FIX | | |
| Effect of Gest. Age | −1.53 FIX | | |
| V1 (mL) | 45.5 (13.76-52.48) | 17.4 (4.6-130) | NE |
| Effect of Weight | 1.0 FIX | | |
| Q (mL/h) | 2.69 (1.55-37.8) | NE | NE |
| Effect of Weight | 0.75 FIX | | |
| V2 (mL) | 109 (67.14-178) | 23.1 (1.2-147) | NE |
| Effect of Weight | 1.0 FIX | | |
| C0int (ug/mL) | 12.3 FIX | 42.8 FIX | NE |
| Effect of Study | 0 FIX | | |
| Ke | 0.000836 FIX | 66.5 FIX | NE |

| | |
|---|---|
| CCV Residual Error | 0 |
| (% CV) | |
| Additive Error | 365 |
| (ug/mL) | |

Graphical comparison of the observed planned and scavenged anti-LTA antibody composition concentrations showed that both types of observations fell within the expected ranges of concentration. However pharmacokinetic model evaluations with the scavenged data caused model instability. Parameter estimates using only planned data were generally more robustly estimated, despite the reduced number of samples available for evaluation. When the pooled data were fit using a separate residual error term for the scavenged samples, the model results for evaluation of all data were generally consistent with the results obtained from the planned data. The fit of only the planned observations, despite having fewer observation records, resulted in parameter estimates that were closer to the previously estimated values and had narrower confidence intervals. The data derived from scavenged samples may not have had appropriate sample times recorded, which would account for the impact of the data when included in the database. Thus, the invention provides that although it is difficult to obtain pharmacokinetic information from pediatric subjects, care must be taken in ensuring that the quality of the data collected are good. The invention further provides that the inclusion of scavenged samples in the database did not improve the parameters estimated.

PK was best described with a linear 2 compartment model. Weight and gestational age were important predictors of clearance. PK parameters were: CL (ml/h) 0.446, V1 75 (ml), V2 138 (ml), t½ 15.4 days. Using the model a dose of 100 mg/kg daily for 3 days followed by weekly infusions of 100 mg/kg for 3 weeks was selected. Observed PK data from 11 VLBW receiving the selected regimen were consistent with model predictions (See FIGS. 8-10). Serum levels were all ≧500 μg/ml, confirming adequate coverage.

Thus, the present invention provides a model-based dose regimen for PAG in VLBW (e.g., to maintain serum antibody levels ≧500 ug/ml). The regimen was prospectively confirmed. PK modeling provided a dosing regimen that could be evaluated with minimal sampling in VLBW.

Planned and scavenged anti-LTA antibody composition concentrations from patients enrolled in clinical trial were evaluated using a two compartment model. Observed concentrations were compared with the expected range of concentrations based on simulations from the original model, and the planned and scavenged data were fit to the same model to evaluate both the performance of the original model and to assess the utility of the scavenged samples. Data were evaluated using Nonmem Version VI level 2 (Icon Inc Hanover Md. USA).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. A method of treating a preterm low birth weight infant comprising providing a preterm low birth weight infant, wherein the infant has, is suspected of having or is at risk for bacterial infection and administering a composition comprising an anti-LTA antibody to the infant under conditions such that the infant obtains a serum concentration of the anti-LTA antibody that is greater than the serum concentration of anti-LTA antibody needed to provide protection from or killing of bacteria in a full term infant.

2. The method of claim 1, wherein the treating is therapeutic.

3. The method of claim 1, wherein the treating is prophylactic.

4. The method of claim 1, wherein the preterm low birth weight infant weighs 2500 grams or less at birth.

5. The method of claim 1, wherein the preterm low birth weight infant weighs 1500 grams or less at birth.

6. The method of claim 1, wherein the preterm low birth weight infant weighs 1000 grams or less at birth.

7. The method of claim 1, wherein the preterm low birth weight infant obtains a serum concentration of anti-LTA antibody of at least 500 μg/ml.

8. The method of claim 7, wherein the preterm low birth weight infant obtains a serum concentration of the anti-LTA antibody of 500 μg/ml within 72 hours of birth.

9. The method of claim 1, wherein the preterm low birth weight infant receives a dose of 600 mg anti-LTA antibody per kilogram infant weight.

10. The method of claim 9, wherein the 600 mg anti-LTA antibody is administered in sequential doses of about 100 mg/kg/day on six separate days.

11. The method of claim 10, wherein the six separate days comprise three consecutive days, days 0, 1, and 2, followed by three non-consecutive days, days 9, 16 and 23.

12. The method of claim 1, wherein the bacterial infection is caused by Gram positive bacteria.

13. The method of claim 1, wherein the composition is administered via intravenous infusion.

14. The method of claim 1, wherein the composition is co-administered with an antibiotic.

15. The method of claim 14, wherein the antibiotic is selected from the group consisting of a β-lactam antibiotic, a penicillin, a cephalosporin, imipenem, monobactams, a β-lactamase inhibitor, vancomycin, an aminoglycoside, a spectinomycin, a tetracycline, chloramphenicol, erythromycin, lincomycin, clindamycin, rifampin, metronidazole, a polymyxin, doxycycline, a quinolone, a sulfonamide, trimethoprim, a quinoline and an anti-staphylococcal antibiotic.

16. The method of claim 15, wherein the co-administration of an anti-LTA binding molecule/anti-LTA antibody and an antibiotic increases the likelihood of survival of the subject from infection compared to the likelihood of survival of the subject from infection if the subject were administered the antibiotic without the anti-LTA binding molecule/anti-LTA antibody.

17. The method of claim 1, wherein the treating is initiated within 12 hours of birth.

18. The method of claim 1, wherein the serum concentration of the anti-LTA antibody that is greater than the serum concentration of anti-LTA antibody needed to provide protection from or killing of bacteria in a full term infant is maintained in the preterm low birth weight infant for three or more days.

19. An anti-LTA antibody composition, wherein the composition is formulated for intravenous infusion of 100 milligrams per kilogram weight of a preterm low birth weight infant.

20. The composition of claim 19, wherein the preterm low birth weight infant weighs 2500 grams or less at birth.

21. The composition of claim 19, wherein the preterm low birth weight infant weighs 1500 grams or less at birth.

22. The composition of claim 19, wherein the preterm low birth weight infant weighs 1000 grams or less at birth.

23. The composition of claim 19, wherein said composition is formulated for intravenous infusion.

24. The composition of claim 19, and a pharmaceutically acceptable carrier.
